(19) United States
(12) Patent Application Publication
(10) Pub. No.: US 2020/0368528 A1
(43) Pub. Date: Nov. 26, 2020

(54) TREATMENT OF EYE DISORDERS

(71) Applicants: GALVANIi BIOELECTRONICS LIMITED, Brentford, Middlesex (GB); UNIVERSITY OF SOUTHERN CALIFORNIA, Los Angeles, CA (US)

(72) Inventors: Arun SRIDHAR, Stevenage (GB); Andrew C WEITZ, Los Angeles, CA (US); Victor Eugene PIKOV, Stevenage (GB); Alessandra GIAROLA, Stevenage (GB); Mark HUMAYUN, Los Angeles, CA (US)

(73) Assignees: Galvani Bioelectronics Limited, Brentford, Middlesex (GB); University of Southern California, Los Angeles (US)

(21) Appl. No.: 16/634,250

(22) PCT Filed: Jul. 27, 2018
(86) PCT No.: PCT/US2018/044206

§ 371 Date: Jan. 27, 2020

Related U.S. Application Data
(60) Provisional application No. 62/538,502, filed on Jul. 28, 2017.

(51) Int. Cl. A61N 1/36; A61N 1/378

(52) U.S. Cl. CPC A61N 1/36046; A61N 1/3787; A61N 1/36121

(57) ABSTRACT

Modulation of neural signaling of an eye-related sympathetic nerve can mitigate choroidal neovascularization (CNV) in the eye, and this provides a way of treating eye disorders, such as ocular neovascular diseases.

### CROSS REFERENCE TO RELATED APPLICATIONS

This application claims the benefit of U.S. Provisional Patent Application No. 62/538,502, filed Jul. 28, 2017.

### TECHNICAL FIELD

This disclosure relates to the treatment of eye disorders, more particularly to methods and medical devices that deliver electromodulation therapy for such purposes.

### BACKGROUND

Ocular neovascular diseases, such as exudative age-related macular degeneration (wet AMD), are the most common cause of moderate to severe vision loss in developed countries [Reference 1]. These diseases are typically treated with intraocular injections of drugs that target VEGF. The release of VEGF is thought to contribute to increased vascular permeability in the eye and inappropriate new vessel growth. The VEGF injections must be given every 4-6 weeks and carry a number of risks. The drugs are effective in slowing disease progression but do not prevent eventual vision loss. Furthermore, traditional anti-VEGF agents are known to promote scarring in wet AMD.

Several lines of evidence suggest a critical role of the sympathetic nervous system in maintaining ocular vascular homeostasis [Reference 2]. Evidence in animal models suggests that a decrease of the βadrenergic function may result in reduction or exacerbation of the vascular changes, thus suggesting possible dual effects of β-adrenoreceptor (β-AR) modulation. There is also evidence suggesting that these vascular changes are associated with changes in the expression and secretion of angiogenic growth factors, such as vascular endothelial growth factor (VEGF) and pigment epithelium-derived factor (PEDF), which are regulated by the sympathetic nerves [References 2, 3, 4]. [1] Campochiaro, P. A., Journal of Molecular Medicine, 91:311-321.[2] Casini, et al., Progress in Retinal and Eye Research, 2014; 42:103-129.[3] Wiley et al., Invest Ophthalmol Vis Sci, 2006; 47(1):439-43.[4] Steinle et al., Exp Eye Res, 2006; 83(1):16-23.

These observations have prompted the use of β-AR blockers in therapy. For example, oral administration of the β1-/β2-AR Worker propranolol in clinical trials in preterm infants with retinopathy of prematurity (ROP) produced positive results in terms of efficacy, although safety problems were also reported. However, there are data demonstrating significant anti-apoptotic effects exerted by β-AR agonists; therefore if β-AR blockers were used to inhibit aberrant neovascularization, there may be a burden to pay in terms of impaired neuronal viability [Reference 2].

The disclosure aims to provide further and unproved treatments of eye disorders, such as eye disorders that are associated with vascular remodeling, e.g. ocular neovascular diseases.

### SUMMARY

The inventors found that modulation of neural activity of an eye-related sympathetic nerve (e.g. the internal carotid nerve (ICN)) is capable of regulating vascular remodeling, so it provides a way to treat eye disorders, such as ocular neovascular diseases. In particular, the inventors found that blocking ocular sympathetic activity (e.g. through ICN denervation or by a local β-AR antagonist) can mitigate choroidal neovascularization (CNV). These findings were made in the laser-induced CNV model, which has become the gold standard for preclinical testing all current treatment modalities used to date for subretinal neovascularization [Reference 5]. Even when ICN denervation was performed 1 week after laser photocoagulation, mitigation of CNV progression was still observed. The results therefore suggest that applying a signal (e.g. an electrical signal) to the ICN to modulate (e.g. inhibit) the neural activity of the ICN could be an effective strategy for treating eye disorders, for example, an eye disorder that is associated with ocular neovascularization, such as subretinal neovascularization (e.g. wet AMD), or an ocular neovascular disease caused by injury to the eye.

Thus, the disclosure provides a method of treating an eye disorder in a subject by reversibly modulating the neural activity of an eye-related sympathetic nerve. A preferred way of reversibly modulating (e.g. inhibiting) the neural activity of the eye-related
[5] Pennesi et al., Molecular of Medicine, 2012; 33:487-509.
   sympathetic nerve neural activity uses a device or system which applies a signal (e.g. an electrical signal) to the eye-related sympathetic nerve.

The disclosure also provides a method of treating an eye disorder in a subject, comprising applying a signal to an eye-related sympathetic nerve in the subject to reversibly modulate (e.g. inhibit) the neural activity of the eye-related sympathetic nerve.

The disclosure provides an implantable device or system according to the disclosure comprising at least one neural interfacing element, such as a transducer, preferably an electrode, suitable for placement on, in, or around an eye-related sympathetic nerve, and a signal generator for generating a signal to be applied to the eye-related sympathetic nerve via the at least one neural interfacing element such that the signal reversibly modulates (e.g. inhibits) the neural activity of the eye-related sympathetic nerve to produce a change, preferably an improvement, in one or more physiological parameters in the subject. The physiological parameters may be one or more of the group consisting of: the level of an angiogenic growth factor in the eye, neovascularization (e.g. retinal, choroidal and/or corneal neovascularization), ocular blood flow, blood pressure, blood oxygenation, the extent of vision impairment, the level of an immune response modulator (e.g. a cytokine) in the eye, the extent of blood vessel leakage in the eye, the amount and/or size of drusen deposits in the eye, the extent of macular edema, the extent of retinal cell death, the level of an oxidative stress marker, and the level of a peroxynitrite marker.

The disclosure also provides a method of treating an eye disorder in a subject, comprising: (i) implanting in the subject a device or system of the disclosure; (ii) positioning a neural interfacing element of the device or system in signaling contact with an eye-related sympathetic nerve in the subject; and optionally (iii) activating the device or system.

Similarly, the disclosure provides a method of reversibly modulating (e.g. inhibiting) neural activity in an eye-related sympathetic nerve in a subject, comprising: (i) implanting in the subject as device or system of the disclosure; (ii) positioning a neural interfacing element in signalling contact with an eye-related sympathetic nerve in the subject; and optionally (iii) activating the device or system.

The disclosure also provides a method of implanting a device or a system of the disclosure in a subject, comprising: positioning a neural interfacing element of the device or system in signaling contact with an eye-related sympathetic nerve in the subject.

The disclosure also provides a device or a system of the disclosure, wherein the device or system is attached to an eye-related sympathetic nerve.

The disclosure also provides the use of a device or system for treating an eye disorder in a subject, by reversibly modulating (e.g. inhibiting) the head activity in an eye-related sympathetic nerve in the subject.

The disclosure also provides a charged particle for use in a method of treating an eye disorder, wherein the charged particle causes reversible depolarization or hyperpolarization of the nerve membrane, such that an action potential does not propagate through the modified nerve.

The disclosure also provides a modified eye-related sympathetic nerve to which a neural interfacing element of the system or device of the disclosure is attached. The neural interfacing element is in signaling contact with the eye-related sympathetic nerve and so the eye-related sympathetic nerve can be distinguished from the eye-related sympathetic nerve in its natural state. Furthermore, the nerve is located in a subject who suffers from, or is at risk of, an eye disorder.

The disclosure also provides a modified eye-related sympathetic nerve, wherein neural activity is reversibly modulated (e.g. inhibited) by applying a signal to the eye-related sympathetic nerve.

The disclosure also provides a modified eye-related sympathetic nerve, wherein the nerve membrane is reversibly depolarized or hyperpolarized by an electric field, such that an action potential does not propagate through the modified eye-related sympathetic nerve.

The disclosure also provides a modified eye-related sympathetic nerve bounded by a nerve membrane, comprising a distribution of potassium and sodium ions movable across the nerve membrane to alter the electrical membrane potential of the nerve so as to propagate an action potential along the nerve in a normal state; wherein at least a portion of the nerve is subject to the application of a temporary external electrical field which modifies the concentration of potassium and sodium ions within the nerve, causing depolarization or hyperpolarization of the nerve membrane, thereby temporarily blocking the propagation of the action potential across that portion in a disrupted state, wherein the nerve returns to its normal state once the external electrical field is removed.

The disclosure also provides a modified eye-related sympathetic nerve obtainable by reversibly modulating (e.g. inhibiting) neural activity of the eye-related sympathetic nerve according to a method of the disclosure.

The disclosure also provides a method of modifying an eye-related sympathetic nerve's activity, comprising a step of applying a signal to the eye-related sympathetic nerve in order to reversibly modulate (e.g. inhibit) the neural activity of the eye-related sympathetic nerve in a subject. Preferably the method does not involve a method for treatment of the human or animal body by surgery. The subject already carries a device or system of the disclosure, which is in signaling contact with the eye-related sympathetic nerve.

The disclosure also provides a method of controlling a device or system of the disclosure, which is in signaling contact with the eye-related sympathetic nerve, comprising a step of sending control instructions to the device or system, in response to which the device or system applies a signal to the eye-related sympathetic nerve.

The disclosure also provides a computer system implemented method, wherein the method comprises applying a signal to an eye-related sympathetic nerve via at least one neural interfacing element, preferably an electrode, such that the signal reversibly modulates the neural activity of the eye-related sympathetic nerve to produce a change in a physiological parameter in the subject, wherein the at least one neural interfacing element is suitable for placement on, in, or around an eye related sympathetic nerve, wherein the physiological parameter is one or more of the group consisting of: the level of an angiogenic growth factor in the eye, neovascularization (e.g. retinal, choroidal or corneal neovascularization), ocular blood flow, blood pressure, blood oxygenation, the extent of vision impairment, the level of an immune response modulator (e.g. a cytokine) in the eye, the extent of blood vessel leakage in the eye, the amount and/or size of drusen deposits in the eye, the extent of macular edema, the extent of retinal cell death, the level of an oxidative stress marker, and the level of a peroxynitrite marker.

### BRIEF DESCRIPTION OF THE DRAWINGS

FIG. 1 is a diagram of the sympathetic and parasympathetic innervation of the eye and lacrimal glands, adapted from [Reference 6]. Sympathetic fibers (S) arise from the superior cervical ganglion (SCG) and travel along the internal carotid artery (IC), then (shown as a dotted line) project to the frontal arteries (FA) and sweat glands (SG). Parasympathetic fibers (PS), originating in the superior salivatory nucleus (SSN), traverse the facial nerve (CrN7) and the greater superficial petrosal nerve (GSP) to join the vidian nerve (VN) and synapse in the sphenopalatine ganglion (SPG); postganglionic fibers then loop back as orbital rami (OR) to the cavernous sinus and internal carotid artery where they form a retro-orbital plexus with sympathetic and trigeminal fibers, before advancing to supply the lacrimal glands (LG) and cutaneous circulation of the forehead. Also shown is the external carotid artery (EC) and the first division of the trigeminal nerve (V1).

FIG. 2 shows a photograph of the surgical procedure showing transection of the left ICN.

FIG. 3 shows graphs of UV spectra of 50 mM isoproterenol (pH=6.5) remaining stable over 14 days. FIG. 3A is a graph form results recorded at day 0; FIG. 3B is a graph from results recorded at 7 days; and, FIG. 3C is a graph from results recorded at 14 days.

FIG. 4 is a series of graphs showing FA scores 14 days after laser photocoagulation in each experimental group (n≥35 lesions per group from 5 animals per group), where four laser burns were made per eye. FIG. 4A shows the as lesion score in each group. Scores in the denervation (ICNx+Vehicle) group were significantly lower than scores in the control (Vehicle) group (P<0.05). Error bars indicate SEM, FIG. 4B is a histogram showing the distribution of FA scores in each group.

FIG. 5 is a series of graphs and photographs showing that β-AR modulation leads to smaller CNV lesions in the rat laser photocoagulation model. FIG. 5A is a graph showing that all 3 treatment groups had statistically smaller lesions than the control (Vehicle) group (n≥
[6] Drummond et al., Brain, 1992; 11(5):1429-1445.
   30 lesions per group from 5 animals per group; *** indicates P<0.001; * indicates P<0.05). Lesions in the propranolol (β antagonist) and ICN transection (ICNx+Vehicle) groups were statistically similar in size (P=0.16). Error bars represent SEM. FIG. 5B and FIG. 5C are images of CNV membranes, stained with FITC-labeled isolectin-B4, showing representative lesions from the control group (FIG. 5B) and ICN transection group (FIG. 5C).

FIG. 6 is a series of photographs (FIG. 6A and FIG. 6C) and FA images (FIG. 6B and FIG. 6B) showing corneal neovascularization in rats receiving propranolol eye drops. Images were taken after 14 days of eye drop therapy.

FIG. 7 is a graph showing choroidal VEGF protein levels 14 days after laser photocoagulation in each experimental group (n=5 animals per group). Twelve laser burns were made in a single eye. VEGF levels in the treatment groups were significantly higher than levels in the control (Vehicle) group. Error bars represent SD. *** indicates P<0.001; ** indicates P<0.01.

FIG. 8 is a series of SD-OCT imaging and 3D reconstructions of a laser-induced CNV lesion with retinal edema. FIG. 8A shows fundus reconstruction from 100 OCT frames. The lesion is the hyperreflective area. The optic disc is visible on the right side of the image. FIG. 8B shows an OCT section (green line in FIG. 8A) through the lesion. FIG. 8C shows a 3D reconstruction in same orientation as FIG. 8A (sagittal view). FIG. 8D shows a 3D reconstruction in same orientation as FIG. 8B (axial view). The arrow in FIG. 8D indicates the lesion.

FIG. 9 is a graph showing FA scores at 3, 7, 10, and 14 days after laser photocoagulation in each experimental group (n≥45 lesions per group from 6 animals per group). Four laser burns were made per eye. Scores in the ICNx (Day 7) group were significantly lower than scores in the Sham (Day 7) group on days 10 and 14 (P<0.001, unpaired t-tests). Error bars indicate SEM.

FIG. 10 is a series of graphs showing ICN denervation leads to smaller CNV lesions in the rat laser photocoagulation model. FIG. 10A shows lesion volumes that were measured with SD-OCT at 3, 7, 10, and 14 days after laser treatment. FIG. 10B shows, following euthanasia on day 14, volumes measured ex vivo with confocal microscopy. By day 14, both ICNx groups had statistically smaller lesions than the sham group (n≥44 lesions per group from 6 animals per group; *** indicates P<0.001). Error bars represent SEM.

FIG. 11 is a graph showing correlation of CNV lesion volumes measured from SD-OCT (horizontal axis) and confocal microscopy (vertical axis), 14 days after laser photocoagulation. Each data point represents a lesion (n=117 lesions). Volumes measured by the two techniques are similar and show good correlation (Spearman's ρ=0.60).

FIG. 12 is a block diagram illustrating elements of a system for performing electrical modulation in an eye-related sympathetic nerve (e.g. the ICN) according to the present disclosure.

### DETAILED DESCRIPTION OF ILLUSTRATIVE EMBODIMENTS

### An Eye-Related Sympathetic Nerve

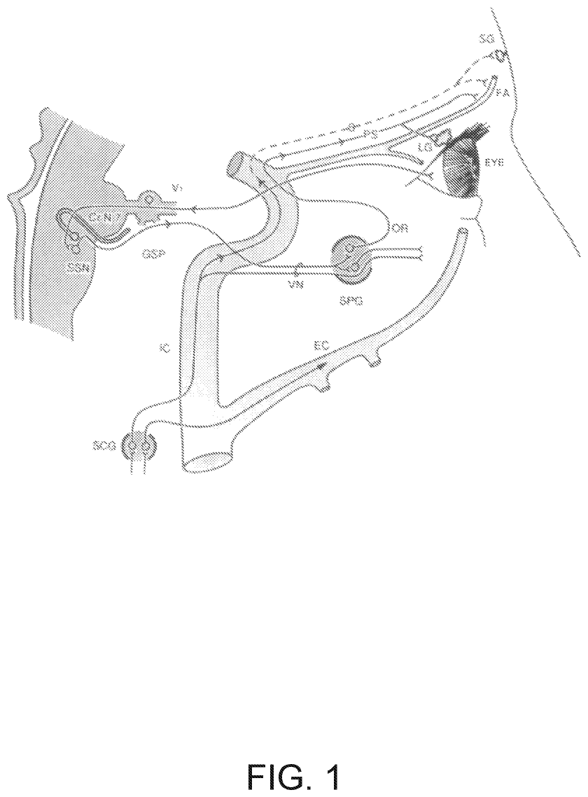

The autonomic nervous system influences numerous ocular functions [Reference 7], including pupil diameter and ocular accommodation, ocular blood flow, and intra-ocular pressure. Sympathetic innervation of the eye arises from preganglionic neurons located in the C8-T2 segments of the spinal cord, a region termed the ciliospinal center of Budge (and Waller). The axons of these preganglionic neurons project to the sympathetic chain ganglia and travel in the sympathetic trunk to the superior cervical ganglion where they contact post ganglionic neurons. The majority of the postganglionic axons leave the superior cervical ganglion through either the external carotid nerve or the internal carotid nerve (ICN). The ICN travels along the internal carotid artery, then projects to the frontal arteries and sweat glands. The ICN is the eye's only source of sympathetic innervation [Reference 8, see FIG. 1].

The superior cervical ganglion lies on the transverse processes of the second and third cervical vertebrae and is possibly formed from four fused ganglia. The internal carotid artery within the carotid sheath is anterior, and longus capitis muscle is posterior. The lower
[7] McDougal and Gamlin, 2015; Compr Physiol.; 5(1):439-473.
[8] Smith et al., Journal of Comparative Neurology, 1990; 301:490-500.
   end of the ganglion is united by a connecting trunk to the middle cervical ganglion. The upper end connects with the ICN [Reference 9].

Postganglionic branches of the superior cervical ganglion are distributed in the ICN, which ascends with the internal carotid artery into the carotid canal to enter the cranial cavity, and in lateral, medial and anterior branches [Reference 9, 10].

The superior cervical ganglion is a consistent structure; human cadaveric studies show that it can be detected in every specimen on both sides [References 10, 11, 12, 13, 14]. One study shows that the common carotid artery bifurcation is a good landmark for localizing the superior cervical ganglion for anaesthetic block [Reference 11]. The data show that the average distance from the inferior pole of superior cervical ganglion to the common carotid artery bifurcation is 4.1 mm (female) and 2.9 mm (male).

Parasympathetic fibers, originating in the superior salivatory nucleus, traverse the facial nerve (CrN7) and the greater superficial petrosal nerve to join the vidian nerve and synapse in the sphenopalatine ganglion, postganglionic fibers then loop back as orbital rami to the cavernous sinus and internal carotid artery where they form a retro-orbital plexus with sympathetic and trigeminal fibers, before advancing to supply the lacrimal glands and cutaneous circulation of the forehead.

Parasympathetic innervation of the eye also originates front neurons in the Edinger-Westphal preganglionic (EWpg) cell group, the autonomic subdivision of the third cranial nerve nucleus, which lies in the rostral mesencephalon. The neurons in EWpg project by way of the oculomotor (III) nerve to postganglionic cells in the ciliary ganglion.
[9] Gray's Anatomy. 41 ed.
[10] Mitsuoka, K., T. Kikutani, and I. Sato, Morphological relationship between the superior cervical ganglion and cervical nerves in Japanese cadaver doctors. Brain Behav, 2017. 7(2): p. e00619.
[11] Wisco, J. J., et. al., A heat map of superior cervical ganglion location relative to the common carotid artery bifurcation. Anesth Analg, 2012. 114(2): p 462-5.
[12] Fazliogullari, Z., et. al., A morphometric analysis of the superior cervical ganglion and its surrounding structures. Surg Radiol Anat, 2016. 38(3): p. 299-302.
[13] Yin, Z., et. al., Neuroanatomy and clinical analysis of the cervical sympathetic trunk and longus colli. J Biomed Res, 2015. 29(6): p. 501-7.
[14] Saylam, C. Y., et al., Neuroanatomy of cervical sympathetic trunk: a cadaveric study. Clin Anat, 2009. 22(3): p. 324-30.

Targets of sympathetic innervation of the eye include blood vessels (e.g. choroidal blood vessels, iris blood vessels, ciliary body blood vessels, episcleral blood vessels). The neural activity of an eye-related sympathetic nerve is naturally associated with the regulation of vascular remodeling in the eye, e.g. altering structure and arrangement in blood vessels through cell growth, cell death, cell migration and/or production or degradation of the extracellular matrix. A potential mechanism for the vascular remodeling may be alterations in the regulation of angiogenic growth factors, e.g. VEGF and PEDF.

Thus, by modulating neural activity in an eye-related sympathetic nerve, it is possible to mitigate choroidal neovascularization (CNV), thereby assisting in treating eye conditions, such as ocular neovascular diseases. For example, inhibition of the neural activity of an eye-related sympathetic nerve can cause reduced choroidal neovascularization, and this could be an effective strategy for treating eye disorders that are associated with subretinal neovascularization, such as wet AMD.

The disclosure can modulate activity at any site along an eye-related sympathetic nerve. For example, the site may be at the cervical portion of the sympathetic trunk, e.g. at the superior cervical ganglion. The site may be at a postganglionic sympathetic nerve projecting from the superior cervical ganglion toward the eye, such as the ICN. Alternatively, the site may be at a preganglionic eye-related sympathetic nerve in the cervical sympathetic trunk.

Preferably, the eye-related sympathetic nerve is modulated at the ICN. The disclosure may modulate at any site along the ICN. For example, the site is in the neck, and e.g. the signal is applied at the ICN in the neck. For example, the site is beneath and/or adjacent to the hypoglossal nerve in the neck. Preferably, the site is amenable for electrodes attachment.

The eye-related sympathetic nerve may be modulated at the superior cervical ganglion. Neuronal subpopulations exist in specific regions of the superior cervical ganglion. For example, the cell bodies of neurons whose axons project out the ICN are located primarily in the rostral part of the superior cervical ganglion [References 15, 16]. The
[15] Li and Horn (2006) J. Neurophysiol 95:187-195.
[16] Bowers and Zigmond (1979) 185: 381-392.
   disclosure preferably modulates these cell bodies. The disclosure preferably modulates the rostral part of the superior cervical ganglion.

Thus, the disclosure may involve applying a signal to an eye-related sympathetic nerve, e.g. the superior cervical ganglion or the cervical portion of the sympathetic trunk, such that all the nerve fibers within the nerve are modulated. Alternatively, the disclosure may involve applying a signal to an eye-related sympathetic nerve, e.g. superior cervical ganglion or the cervical portion of the sympathetic trunk, such that only a portion (e.g. spatial selection) of the nerve fibers and/or cell bodies within the nerve are modulated. The disclosure may additionally involve a step of selecting eye-related sympathetic nerve fibers prior to applying a signal. Methods of selective modulation of nerve fibers within a nerve are known in the art (e.g. see [References 17, 18, 19]).

Where the disclosure refers to a modified eye-related sympathetic nerve, this nerve is ideally present in situ in as subject.

### Modulation of Neural Activity

According to the disclosure, applying a signal (e.g. an electrical signal) to an eye-related sympathetic nerve results in neural activity in at least part of the nerve being modulated. Modulation of neural activity, as used herein, is taken to mean that the signaling activity of the nerve is altered from the baseline neural activity—that is, the signaling activity of the nerve in the subject prior to any intervention. Such modulation may inhibit, block or otherwise change the neural activity compared to baseline activity. As used herein, “neural activity” of a nerve means the signaling activity of the nerve, for example the amplitude, frequency and/or pattern of action potentials in the nerve. The term “pattern”, as used herein in the context of action potentials in the nerve, is intended to include one or more of: local field potential(s), compound action potential(s), aggregate action potential(s), and also magnitudes, frequencies, areas under the curve and other patterns of action potentials in the nerve or sub-groups (e.g. fascicules) of neurons therein.
[17] Accornero et al., J. Physiol. (1977), 273: 39-560.
[18] Ayres et al., J Neurophysiol. 116: 51-60 (2016).
[19] Bruns et al. (2015) Neurology and Urodynamics 34:65-71.

One advantage of the disclosure is that modulation of neural activity is reversible. Hence, the modulation of neural activity is not permanent. For example, upon cessation of the application of a signal, neural activity in the nerve returns substantially towards baseline neural activity within 1-60 seconds, or within 1-60 minutes, or within 1-24 hours (e.g. within 1-12 hours, 1 -6 hours, 1-4 hours, 1-2 hours), or within 1-7 days (e.g. 1-4 days, 1-2 days). In some instances of reversible modulation, the neural activity returns substantially fully to baseline neural activity. That is, the neural activity following cessation of the application of a signal is substantially the same as the neural activity prior to a signal being applied. Hence, the nerve or the portion of the nerve has regained its normal physiological capacity to propagate action potentials.

In other embodiments, modulation of the neural activity may be substantially persistent. As used herein, “persistent” is taken to mean that the modulated neural activity has a prolonged effect. For example, upon cessation of the application of a signal, neural activity in the nerve remains substantially the same as when the signal was being applied—i.e. the neural activity during and following signal application is substantially the same. Reversible modulation is preferred.

The disclosure preferably involves inhibition of neural activity. According to the disclosure, inhibition results in neural activity in at least part of an eye-related sympathetic nerve being reduced compared to baseline neural activity in that part of the nerve. This reduction in activity can be across the whole nerve, in which case neural activity is reduced across the whole nerve. Thus inhibition may apply to both afferent and efferent fibers of an eye-related sympathetic time, but in some embodiments inhibition may apply only to afferent fibers or only to efferent fibers. Preferably the inhibition applies only to efferent fibers.

Inhibition of neural activity may be partial inhibition. Partial inhibition may be such that the total signaling activity of the whole nerve is partially reduced, or that the total signaling activity of a subset of nerve fibers of the nerve is fully reduced (i.e. there is no neural activity in that subset of fibers of the nerve), or that the total signaling of a subset of nerve fibers of the nerve is partially reduced compared to baseline neural activity in that subset of fibers of the nerve. Inhibition of neural activity encompasses full inhibition of neural activity in the nerve—that is, embodiments where there is no neural activity in the whole nerve.

In some cases, the inhibition of neural activity may be a block of neural activity i.e. action potentials are blocked from travelling beyond the point of the block in at least a part of the nerve. A block on neural activity is thus understood to be blocking neural activity from continuing past the point of the block. That is, when the block is applied, action potentials may travel along the nerve or subset of nerve fibers to the point of the block, but not beyond the point of the block. Thus, the nerve at the point or block is modified in that the nerve membrane is reversibly depolarized or hyperpolarized by an electric field, such that an action potential does not propagate through the modified nerve. Hence, the nerve at the point of the block is modified in that it has lost its capacity to propagate action potentials, whereas the portions of the nerve before and after the point of block have the capacity to propagate action potentials.

When an electrical signal is used with the disclosure, the block is based on the influence of electrical currents (e.g. charged particles, which may be one or more electrons in an electrode attached to the nerve, or one or more ions outside the nerve or within the nerve, for instance) on the distribution of ions across the nerve membrane.

At any point along the axon, a functioning nerve will have a distribution of potassium and sodium ions across the nerve membrane. The distribution at one point along the axon determines the electrical membrane potential of the axon at that point, which in turn influences the distribution of potassium and sodium ions at an adjacent point, which in turn determines the electrical membrane potential of the axon at that point, and so on. This is a nerve operating in is normal state, wherein action potentials propagate from point to adjacent point along the axon, and which can be observed using conventional experimentation. One way of characterizing a block of neural activity is a distribution of potassium and sodium ions at one or more points in the axon which is created not by virtue of the electrical membrane potential at adjacent a point or points of the nerve as a result of a propagating action potential, but by virtue of the application of a temporary external electrical field. The temporary external electrical field artificially modifies the distribution of potassium and sodium ions within a point in the nerve, causing depolarization or hyperpolarization of the nerve membrane that would not otherwise occur. The depolarization or hyperpolarization of the nerve membrane caused by the temporary external electrical field blocks the propagation of an action potential across that point, because the action potential is unable to influence the distribution of potassium and sodium ions, which is instead governed by the temporary external electrical field. This is a nerve operating in a disrupted state, which can be observed by a distribution of potassium and sodium ions at a point in the axon (the point which has been blocked) that has an electrical membrane potential that is not influenced or determined by a the electrical membrane potential of an adjacent point.

Blocking may be a partial block. Partial block may be such that the total signaling of a subset of nerve fibers of the nerve is partially reduced compared to baseline neural activity in that subset of fibers of the nerve. For example a reduction in neural activity 5%, 10%, 15%, 20%, 25%, 30%, 35%, 40%, 45%, 40%, 50%, 60%, 70%, 80%, 90% or 90% or 95%, or blocking of neural activity in a subset of nerve fibers of the nerve. The neural activity may be measured by methods known in the art, for example, by the number of action potentials which propagate through the axon and/or the amplitude of the local field potential reflecting the summed activity of the action potentials. Block of neural activity may be a full block, i.e. blocking of neural activity in the whole nerve.

The disclosure may selectively block nerve fibers of various sizes within a nerve. Larger nerve fibers tend to have a lower threshold for blocking than smaller nerve fibers. Thus, for example, increasing signal amplitude (e.g. increasing amplitude of art electric signal) may generate block of the smaller fibers. Methods of selective modulation of nerve fibers within a nerve are known in the art (e.g. see [References 17, 18, 19]).

Modulation of neural activity may be an alteration in the pattern of action potentials. It will be appreciated that the pattern of action potentials can be modulated without necessarily changing the overall frequency or amplitude. For example, modulation of neural activity may be such that the pattern of action potentials is altered to more closely resemble a healthy state rather than a disease state.

Modulation of neural activity may comprise altering the neural activity in various other ways, for example decreasing a particular part of the neural activity and/or reducing new elements of activity, for example: in particular intervals of time, in particular frequency bands, according to particular patterns and so forth.

Modulation of neural activity may be (at least partially) corrective. As used herein, “corrective” is taken to mean that the modulated neural activity alters the neural activity towards the pattern of neural activity in a healthy subject, and this is called axonal modulation therapy. That is, upon cessation of sing al application, neural activity in the nerve more closely resembles (ideally, substantially fully resembles) the pattern of action potentials in the nerve observed in a healthy subject-than prior to signal application. Such corrective modulation can be any modulation as defined herein. For example, application of a signal may result in a block on neural activity, and upon cessation of signal application the pattern of action potentials in the nerve resembles the pattern of action potentials observed in a healthy subject. By way of further example, application of the signal may result in neural activity resembling the pattern of action potentials observed in a healthy subject and, upon cessation of the signal, the pattern of action potentials in the nerve remains the pattern of action potentials observed in a healthy subject.

### Eye Disorders

The disclosure is useful in treating an eye disorder. For example, the disclosure is useful in slowing, stopping or reversing progression of an eye disorder, such as an ocular neovascular disease.

The disclosure is particularly useful for treating eye disorders that are associated with ocular neovascularization, such as subretinal neovasoularization. For example, the disclosure is useful for treating an eye disorder that is caused by or associated with the growth of blood vessels and/or blood vessel leakage in the eye. The disclosure may also be useful for treating eye disorders that have an imbalance of angiogenic growth factors compared to the physiological homeostatic state.

The disclosure may also be useful for treating an ocular neovascular disease caused by injury to the eye, e.g. by applying a signal (e.g. an electrical signal) to modulate (e.g. inhibit) the neural activity of an eye-related sympathetic nerve. For example, the eye injury may be a retinal injury, a corneal injury or conjunctival injury. The eye injury may be caused by trauma, e.g. surgical injuries, chemical burn, corneal transplant, infectious or inflammatory diseases.

The disclosure is particularly useful in treating the development of exudative age-related macular degeneration (wet AMD), e.g. by applying a signal (e.g. an electrical signal) to modulate (e.g. inhibit) the neural activity of an eye-related sympathetic nerve. The most common cause of decreased best-corrected vision in subjects over 65 years of age in the US is the retinal disorder known as age-related macular degeneration (AMD). As AMD progresses, the disease is characterized by loss of sharp, central vision. The area of the eye affected by AMD is the macula—a small area in the center of the retina, composed primarily of photoreceptor cells. So-called “dry” AMD, accounting for about 85%-90% of subjects with AMD, involves alterations in eye pigment distribution, loss of photoreceptors and diminished retinal function due to overall atrophy of cells. So-called “wet” AMD involves proliferation of abnormal choroidal vessels leading to clots or scars in the sub-retinal space. Thus, the onset of wet AMD occurs because of the formation of an abnormal choroidal neovascular network (choroidal neovascularization, CNV) beneath the neural retina. The newly formed blood vessels are excessively leaky. This leads to accumulation of subretinal fluid and blood leading to loss of visual acuity. Eventually, there is total loss of functional retina in the involved region, as a large disciform scar involving choroids and retina forms. While subjects with dry AMD may retain vision of decreased quality, wet AMD often results in blindness.

Dry AMD typically presents three main stages. In the early stage, there may be many small collections of drusen (deposits) inside the eye (e.g. <63 microns in diameter), a few medium-sized drusen (e.g. 63-124 microns in diameter), or some minor damage to the retina; early AMD may not cause any noticeable symptoms. In the intermediate stage, there may be some larger drusen inside the eye (e.g. ≥125 microns in diameter) or some tissue damage to the outer section of the macula. The subject will typically have a blurred spot in the center of his vision. In the advanced stage, the center of the macula is damaged. The subject will typically have a large blurred central spot and find it difficult to read and recognize faces. Wet AMD is typically considered to be an advanced form of AMD. In the more progressive stage of wet AMD, the subject has serious visual impairment.

The disclosure may be useful in preventing progression of AMD, e.g. by applying a signal (e.g. an electrical signal) to modulate (e.g. inhibit) the neural activity of an eye-related sympathetic nerve. For example, the disclosure may be useful in preventing the progression from dry AMD to wet AMD. The disclosure may be useful in preventing the progression from an early stage of wet AMD to a more progressive stage of wet AMD, e.g. by applying a signal (e.g. an electrical signal) to modulate (e.g. inhibit) the neural activity of an eye-related sympathetic nerve.

The disclosure is also useful for treating disorders associated with CNV, e.g. by applying a signal (e.g. an electrical signal) to modulate (e.g. inhibit) the neural activity of an eye-related sympathetic nerve. CNV occurs not only in wet AMD but also in other ocular pathologies such as ocular hismplasmosis syndrome, angiod streaks, ruptures in Bruch's membrane, myopic degeneration, ocular tumors and some retinal degenerative diseases.

The disclosure may also be useful for treating central retinal vein occlusion (CRVO), e.g. by applying a signal (e.g. an electrical signal) to modulate (e.g. inhibit) the neural activity of an eye-related sympathetic nerve. CRVO is caused by obstruction of the central retinal vein that leads to a back-up of blood and fluid in the retina. The retina can also become ischemic, resulting in the growth of new, inappropriate blood vessels that can cause further vision kiss and more serious complications.

A subject of the disclosure may, in addition to having an implant, receive medicine for their eye condition. For instance, a subject having an implant according to the disclosure may receive an anti-VEGF agent, e.g. an anti-VEGF antibody such as ranibizumab (which will usually continue medication which was occurring before receiving the implant). Thus the disclosure provides the use of these medicines in combination with a device or system of the disclosure.

A subject suitable for the disclosure may be any age, but will usually be at least 55, 60, 65, 70, 75, 80 or 85 years of age.

### Physiological Parameters

Treatment of an eye disorder can be assessed in various ways, but typically involves determining an improvement in one or more physiological parameters of the subject. As used herein, an “improvement in a determined physiological parameter” is taken to mean that, for any given physiological parameter, an improvement is a change in the value of that parameter in the subject towards the normal value or normal range for that value—i.e. towards the expected value in a healthy subject.

As used herein, worsening of a determined physiological parameter is taken to mean that, for any given physiological parameter, worsening is a change in the value of that parameter in the subject away from the normal value or normal range for that value—i.e. away from the expected value in a healthy subject.

Useful physiological parameters of the disclosure may be one or more of the group consisting of: the level of an angiogenic growth factor in the eye, neovascularization (e.g. retinal, choroidal and or corneal neovascularization), ocular blood flow, blood pressure, blood oxygenation, the extent of vision impairment, the level of an immune response modulator (e.g. a cytokine) in the eye, the extent of macular edema, the extent of blood vessel leakage in the eye, the amount and/or size of drusen deposits in the eye, the extent of retinal cell death, the level of an oxidative stress marker, and the level of a peroxynitrite marker.

For example, in a subject having an eye disorder associated with subretinal ocular neovascularization, such as wet AMD or an ocular neovascular disease caused by injury to the eye, an improvement in a physiological parameter may (depending on which abnormal values a subject is exhibiting) be one or more of the group consisting of: a decrease in the level of a pro-angiogenic growth factor in the eye, an increase in the level of an anti-angiogenic growth factor in the eye, a decrease in choroidal neovascularization (CNV), a decrease in macular edema, a decrease in blood vessel leakage in the eye, a decrease in the number of drusen deposits and/or size thereof, an improvement in central vision, a decrease in retinal cell death, an increase in blood oxygenation, a decrease in the level of an oxidative stress marker, and a decrease in the level of a peroxynitrite marker. The disclosure might not lead to a change in all of these physiological parameters.

The disclosure preferably causes regression of the CNV lesions, stabilizing the CNV lesion, and/or preventing progression of an active CNV lesion.

Suitable methods for determining the value for one or more physiological parameter will be appreciated by the skilled person. By way of example, central vision may be assessed by the Amsler Grid test. Retinal imaging is a typical way for identifying changes in the retina and macula. Commonly used retinal imaging techniques are color fundus photography, fluorescein angiography (FA), indocyanine green angiography (ICGA), optical coherence tomography (OCT), and fundus autofluorescence (FAF). For example, retinal imaging techniques can identify whether the macula is thickened or abnormal, and whether any fluid has leaked into the retina.

The disclosure may increase the levels of anti-inflammatory cytokines in the eye, and/or decrease the levels of pro-inflammatory cytokines in the eye. Ways to measure the levels of these cytokines are known in the art. For example, the protein levels of these cytokines may be measured in a sample from the subject, e.g. in the aqueous humor of the eye, with ELISA.

Pro-inflammatory cytokines are known in the art. Examples of these include tumor necrosis factor (TNF; also known as TNF-α or cachectin), interleukin (IL)-1α, IL-1β, IL-2, IL-5, IL-8, IL-15, IL-18, interferon γ (IFN-γ), platelet-activating factor (PAF), thromboxane, soluble adhesion molecules, vasoactive neuropeptides, phospholipase A2, plasminogen activator inhibitor (PAI-1), free radical generation; neopterin, CD14, prostacyclin, neutrophil elastase, protein kinase, monocyte chemotactic proteins 1 and 2 (MCP-1, MCP-2), macrophage migration inhibitory factor (MIF), high mobility group box protein 1 (HMGB-1), and other known factors. Anti-inflammatory cytokines are also known in the art. Examples of these include IL-4, IL-10, IL-17, IL-13, IL-1α, and TNF-α receptor. It will be recognized that some of pro-inflammatory cytokines may act as anti-inflammatory cytokines in certain circumstances, and vice-versa. Such cytokines are typically referred to as pleiotropic cytokines.

For example, inflammatory cytokines such as C-reactive protein, homocysteine, and plasma complement activation fragments may accelerate progression to advanced AMD. The disclosure therefore preferably reduces the levels of any of these pro-inflammatory cytokines, for example, by applying a signal (e.g. an electrical signal) to modulate (e.g. inhibit) an eye-related sympathetic nerve (e.g. the ICN).

The disclosure preferably decreases the levels of pro-angiogenic growth factors, such as vascular endothelial growth factor (VEGF), e.g. VEGF-A, and/or increases the levels of anti-angiogenic growth factors, such as pigment epithelial-derived factor (PEDF). PEDF is anti-angiogenic at low doses, but pro-angiogenic at high doses [Reference 20]. For example, applying a signal (e.g. an electrical signal) to modulate (e.g. inhibit) an eye-related sympathetic nerve (e.g. the ICN) may cause these changes.

Oxidative stress markers and peroxynitrite markers, and methods of measuring the levels of these markers, are well known in the art (e.g. see references 21, 22).

In certain embodiments of the disclosure, treatment of the condition is indicated by an improvement in the profile of neural activity in the eye-related sympathetic nerve. That is, treatment of the condition is indicated by the neural activity in the eye-related sympathetic nerve approaching the neural activity in a healthy subject.

As used herein, a physiological parameter is not affected by modulation of the neural activity of the eye-related sympathetic nerve if the parameter does not change (in response to the eye-related sympathetic nerve activity modulation) from the normal value or normal range for that value of that parameter exhibited by the subject or subject when no intervention has been performed i.e. it does not depart from the baseline value for that parameter.

Preferably, modulation of the neural activity of the eye-related sympathetic nerve has minimal impact on pupil diameter. More preferably, modulation of the neural activity of the eye-related sympathetic nerve does not produce a change in pupil diameter. Changes in pupil diameter (e.g. the extent of pupil constriction) may thus be a useful indicator for optimization of the parameters of the system or device of the disclosure. If pupil diameter is affected, the methods of the disclosure could be applied while the subject is asleep.

The skilled person will appreciate that the baseline for any neural activity or physiological parameter in an subject need not be a fixed or specific value, but rather can fluctuate within a normal range or may be an average value with associated error and confidence intervals. Suitable methods for determining baseline values are well known to the skilled person.
[20] R. S. Apte et al., Investigative Ophthalmology & Visual Science, vol. 45, pp. 4491-4497, 2004
[21] Blasiak et al., BioMed Research International (2014) 768026
[22] Chiou, (2001) J. Ocul. Pharmacol. Ther. (2):189-98.

As used herein, a physiological parameter is determined in a subject when the value for that parameter exhibited by the subject at the time of detection is determined. A detector (e.g. a physiological sensor subsystem, a physiological data processing module, a physiological sensor, etc.) is any element able to make such a determination.

Thus, in certain embodiments, the disclosure further comprises a step of determining one or more physiological parameters of the subject, wherein the signal is applied only when the determined physiological parameter meets or exceeds a predefined threshold value. In such embodiments wherein more than one physiological parameter of the subject is determined, the signal may be applied when any one of the determined physiological parameters meets or exceeds its threshold value, alternatively only when all of the determined physiological parameters meet or exceed their threshold values. In certain embodiments wherein the signal is applied by a device or system of the disclosure, the device or system lather comprises at least one detector configured to determine the one or more physiological parameters of the subject.

In certain embodiments, the physiological parameter is an action potential or pattern of action potentials in a nerve of the subject, wherein the action potential or pattern of action potentials is associated with the condition that is to be treated. For example, the nerve is the eye-related sympathetic nerve. In this embodiment, the pattern of action potentials determined by the at least one detector may be associated with an eye disorder.

It will be appreciated that any two physiological parameters may be determined in parallel embodiments, the controller is coupled detect the pattern of action potentials tolerance in the subject.

A “predefined threshold value” for a physiological parameter is the minimum (or maximum) value for that parameter that must be exhibited by a subject or subject before the specified intervention is applied. For any given parameter, the threshold value may be defined as a value indicative of a pathological state or a disease state (e.g. the blood oxygenation level in the eye is greater than a threshold level, or greater than the blood oxygenation level in the eye of a healthy subject). The threshold value may be defined as a value indicative of the onset of a pathological state or a disease state. Thus, depending on the predefined threshold value, the disclosure can be used as a treatment. Alternatively, the threshold value may be defined as a value indicative of a physiological state of the subject (that the subject is, for example, asleep, post-prandial, or exercising). Appropriate values for any given physiological parameter would be simply determined by the skilled person (for example, with reference to medical standards of practice).

Such a threshold value for a given physiological parameter is exceeded if the value exhibited by the subject is beyond the threshold value—that is, the exhibited value is a greater departure from the normal or healthy value for that physiological parameter than the predefined threshold value.

### An Implantable Device or System for Implementing the Disclosure

An implantable system according to the disclosure comprises an implantable device (e.g. implantable device 106 of FIG. 12). The implantable device comprises at least one neural interfacing element such as a transducer, preferably an electrode (e.g. electrode 108), suitable for placement on, in, or around an eye-related sympathetic nerve. The implantable system preferably also comprises a processor (e.g. microprocessor 113) coupled to the at least one neural interfacing element.

The at least one neural interfacing element may take many forms, and includes any component which, when used in an implantable device or system for implementing the disclosure, is capable of applying a stimulus or other signal that modulates electrical activity, e.g., action potentials, in a nerve.

The various components of the implantable system are preferably part of a single physical device, either sharing a common housing or being a physically separated collection of interconnected components connected by electrical leads (e.g. leads 107). As an alternative, however, the disclosure may use a system in which the components are physically separate, and communicate wirelessly. Thus, for instance, the at least one neural interfacing element (e.g. electrode 108) and the implantable device (e.g. implantable device 106) can be part of a unitary device, or together may form an implantable system (e.g. implantable system 116). In both cases, further components may also be present to form a larger device or system (e.g. system 100).

### Suitable Forms of a Modulating Signal

The disclosure uses a signal applied via one or more neural interfacing elements (e.g. electrode 108) placed in signaling contact with an eye-related sympathetic nerve (e.g. the ICN).

Signals applied according to the disclosure are ideally non-destructive. As used herein, a “non-destructive signal” is a signal that, when applied, does, not irreversibly damage the underlying neural signal conduction ability of the nerve. That is, application of a non-destructive signal maintains the ability of the nerve (e.g. an eye-related sympathetic nerve) or fibers thereof, or other nerve tissue to which the signal is applied, to conduct action potentials when application of the signal ceases, even if that conduction is in practice artificially stimulated as a result of application of the non-destructive signal.

The signal will usually be an electrical signal, which may be, for example, a voltage or current waveform. The at least one neural interfacing element (e.g. electrode 108) of the implantable system (e.g. implantable system 116) is configured to apply the electrical signals to a nerve, or a part thereof. However, electrical signals are just one was of implementing the disclosure, as is further discussed below.

An electrical signal can take various forms, for example, a voltage or current. In certain such embodiments the signal applied comprises a direct current (DC), such as a charge-balanced DC, or a charge-balanced alternating current (AC) waveform, or both a DC and an AC waveform. A combination of charge-balanced DC and AC is particularly useful, with the DC being applied for a short initial period after which only AC is used [Reference 23]. As used herein, “charge-balanced” in relation to a DC current is taken to mean that the positive or negative charge introduced into any system (e.g. a nerve) as a result of a DC current being applied is balanced by the introduction of the opposite charge in order to achieve overall (net) neutrality. In other words, a charge-balance DC current includes a cathodic pulse and an anodic pulse.

In certain embodiments, the DC waveform or AC waveform may be a square, sinusoidal, triangular, trapezoidal, quasitrapezodial or complex waveform. The DC waveform
[23] Franke et al. J Neural Eng. 2014; 11(5):056012.
   may alternatively be a constant amplitude waveform. In certain embodiments the electrical signal is an AC sinusoidal waveform. In other embodiments, the waveform comprises one or more pulse trains, each comprising a plurality of charge-balanced biphasic pulses.

The signal may be applied in bursts. The range of burst durations may be from seconds to hours; applied continuously in a duty cycled manner from 0.01% to 100%, with a predetermined time interval between bursts. The electric signal may be applied as step change or as a ramp change in current or intensity. Particular signal parameters for modulating (e.g. inhibiting) an eye-related sympathetic nerve are further described below.

Modulation of the neural activity of the eye-related sympathetic nerve can be achieved using electrical signals which serve to replicate the normal neural activity of the nerve. Inhibition or blocking of neural activity of the eye-related sympathetic nerve may be realized using any form of block. For example, by application of one or more of: a DC block, AC block, HFAC block, KHFAC block, anodal block or any other block known in the art.

With reference again to FIG. 12, the implantable system 116 comprises an implantable device 106 which may comprise a signal generator 117 (not shown); for example, a pulse generator. When the implantable device comprises a pulse generator, the implantable device 106 may be referred to as an implantable pulse generator. The signal generator 117 may also be a voltage or current source. The signal generator 117 may be pre-programmed to deliver one or more pre-defined waveforms with signal parameters falling within the range given below. Alternatively, the signal generator 117 may be controllable to adjust one or more of the signal parameters described further below. Control may be open loop, wherein the operator of the implantable device 106 may configure the signal generator using an external controller (e.g. controller 101), or control may be closed loop, wherein signal generator modifies the signal parameters in response to one or more physiological parameters of the subject, as is further described below.

### Signal Parameters for Modulating Neural Activity

In all of the above examples, the signal generator 117 may be configured to deliver an electrical signal for modulating (e.g. inhibiting) an eye-related sympathetic nerve (e.g. the ICN). In the present application, the signal generator 117 is configured to apply an electrical signal with certain signal parameters to modulate (e.g. inhibit) neural activity in an eye-related sympathetic nerve (e.g. the ICN). Signal parameters for modulating (e.g. inhibiting) the eye-related sympathetic nerve, which are described herein, may include waveform, amplitude and frequency.

In certain embodiments for inhibiting neural activity in an eye-related sympathetic nerve, the electrical signal has a frequency of 0.5 to 100 kHz, optionally 1 to 50 kHz, optionally 5 to 50 kHz. In certain embodiments for inhibiting neural activity, the signal has a frequency of 25 to 55 kHz, optionally 30 to 50 kHz. In other embodiments for inhibiting neural activity, the signal has a frequency of 5 to 10 kHz. In certain embodiments for inhibiting neural activity, the electrical signal has a frequency of greater than 1 kHz. In certain embodiments for inhibiting neural activity, the electrical signal has a frequency of greater than 20 kHz, optionally at least 25 kHz, optionally at least 30 kHz. In certain embodiments the signal has a frequency of 30 kHz, 40 kHz, or 50 kHz.

The signal generator 117 may be configured to deliver one or more pulse trains at intervals according to the above-mentioned frequencies. For example, a frequency of 1 to 50 Hz results in a pulse interval between 1 pulse per second and 50 pulses per second, within a given pulse train. The range of pulse widths may be from 0.01 to 2 ms (including, if applicable, both positive and negative phases of the pulse, in the case of a charge-balanced biphasic pulse). The range of pulse amplitudes may be from 0.01 to 10 mA peak-to-peak.

In certain embodiments for inhibiting neural activity in an eye-related sympathetic nerve, the electrical signal has a current of 0.1 to 10 mA, optionally 0.5 to 5 mA, optionally 1 mA to 2 mA, optionally 1 mA or 2 mA.

In certain embodiments for inhibiting neural activity in an eye-related sympathetic nerve, the signal is an electrical signal comprising an AC sinusoidal waveform having a frequency of greater than 25 kHz, optionally 30 to 50 kHz. In certain such embodiments, the signal can be an electrical signal comprising an AC sinusoidal waveform having a frequency of greater than 25 kHz, optionally 30 to 50 kHz, having a current of 1 mA or 2 mA.

Some useful electrical signals for inhibiting neural activity in an eye-related sympathetic nerve may be direct current (DC) or alternating current (AC) waveforms applied to the nerve using one or more electrodes (e.g. electrode 108). A DC block may be accomplished by gradually ramping up the DC waveform amplitude [Reference 24].

Some other AC techniques for inhibiting neural activity in an eye-related sympathetic nerve include high-frequency alternating current (HFAC), or kilohertz-frequency alternating current (KHFAC) to which provides a reversible block. For example, a sinusoidal or rectangular waveform at 3 to 5 kHz (HFAC), and typical signal amplitudes that produced block were 3 to 5 Volts, or 0.5 to 2.0 mA peak-to-peak [Reference 25]. Further details of charge-balanced KHFAC for the blocking of neural activity, which can be used with the disclosure, are discussed in [Reference 26]. Advantageously, the blocking in KHFAC is reversible.

KHFAC may typically be applied at a frequency of between 1 and 50 kHz at a duty cycle of 100% [Reference 27]. Methods for selectively blocking activity of a nerve by application of a waveform having a frequency of 5 to 10 kHz are described in [Reference 28]. Similarly, [Reference 29] describes a method of ameliorating sensory nerve pain by applying a 5 to 50 kHz frequency waveform to a nerve.

When applying a KHFAC signal, there may be a short period in which the nerve is stimulated (an “onset response” or “onset effect”). Various ways of avoiding an onset response are available. In certain embodiments, an onset response as a result of the signal being applied can be avoided if the signal does not have a frequency of 20 kHz or lower, for example 1 to 20 kHz, or 1 to 10 kHz. Frequency- and amplitude-transitioned waveforms can also mitigate onset responses in high-frequency nerve blocking [Reference 30]. Amplitude ramping may also be used [31], or a combination of KHFAC with charge balanced direct
[24] Bharha & Kilgore, IEEE Transactions on Neural systems and rehabilitation engineering, 2004 12:313-324.
[25] Kilgore & Bhadra, Medical and Biological Engineering and Computing, 2004; 42(3):394-406
[26] Kilgore & Bhadra, Neuromodulation, 2014; 17:242-55.
[27] Bhadra et al., Journal of Computational Neuroscience, 2007, 22:313-326.
[28] U.S. Pat. No. 7,389,145.
[29] U.S. Pat. No. 8,731,676.
[30] Gerges et al., J. Neural Eng., 2010; 7:066003.
[31] Bhadra et al., IEMBS, 2009; 5332735.
   current waveforms may be used [Reference 32]. A combination of KHFAC and infra-red laser light (ACIR) may also be used to avoid onset responses [Reference 33].

It will be appreciated by the skilled person that the current amplitude of an applied electrical signal necessary to achieve the intended modulation of the neural activity will depend upon the positioning of the electrode and the associated electrophysiological characteristics (e.g. impedance). It is within the ability of the skilled person to determine the appropriate current amplitude for achieving the intended modulation of the neural activity in a given subject.

### Electrodes

As mentioned above, the implantable system comprises at least one neural interfacing element, the neural interfacing element is preferably art electrode 108. The neural interface is configured to at least partially and preferably fully circumvent the eye-related sympathetic nerve. The geometry of the neural interface is defined in part by the anatomy of the eye-related sympathetic nerve. In particular, the geometry may be limited by the length of the eye-related sympathetic nerve and/or by the diameter of the eye-related sympathetic nerve. For example, the dimensions of the ganglia useful with the disclosure are shown in Table 1.

TABLE 1
Measurements of the superior cervical ganglion, single
middle cervical ganglion and the inferior cetvical/cervicothoracic
ganglion [Reference 34].

| | | Mean | Min. | Max. |
|---|---|---|---|---|
| | | (mm) | (mm) | (mm) |
| Superior cervical ganglion | Length | 33.0 ± 6.2 | 13.1 | 45.7 |
| | Width | 8.1 ± 5.4 | 3.8 | 17.6 |
| Single middle cervical ganglion | Length | 8.9 ± 5.4 | 3.0 | 21.6 |
| | Width | 5.1 ± 2.1 | 2.9 | 9.6 |
| Inferior cervical/ | Length | 11.3 ± 4.6 | 5.1 | 23 |
| cervicothoracic ganglion | Width | 8.2 ± 3.0 | 3.5 | 15.6 |

[32] Franke et al., J Neural Eng., 2014; 11(5):056012.
[33] Lothet et al., Neurophotonics, 2014; 1(1):011010.

In some embodiments (for example, FIG. 12), electrode 108 may be coupled to implantable device 106 of implantable system 116 is electrical leads 107. Alternatively, implantable device 106 may be directly integrated with the electrode 108 without leads. In any case, implantable device 106 may comprise DC current blocking output circuits, optionally based on capacitors and/or inductors, on all output channels (e.g. outputs to the electrode 108, or physiological sensor 111). Electrode 108 may be shaped as one of: a rectangle, an oval, an ellipsoid, a rod, a straight wire, a curved wire, a helically wound wire, a barb, a hook, or a cuff. In addition to electrode 108 which, in use, is located on, in, or near an eye-related sympathetic nerve (e.g. the ICN), there may also be a larger indifferent electrode placed 119 (not shown) in the adjacent tissue.

Preferably, electrode 105 may contain at least two electrically conductive exposed contacts 109 configured, in use, to be placed on, in, or near an eye-related sympathetic nerve to innervate the eye. Exposed contacts 109 may be positioned, in use, transversely along the axis of an eye-related sympathetic nerve. In this configuration, the distance between each of the at least two exposed contacts may be between about 0.5 mm and about 5 mm, optionally between about 1 mm and 3 mm, optionally between about 1 mm and 2 mm. Each of the at least two exposed contacts 109 may have a surface area in contact with an eye-related sympathetic nerve which is equal to that of the other. The surface area may range between about 0.1 mm² and about 100 mm², optionally between about 1 mm² to 50 mm², optionally between about 1 mm² to 20 mm², optionally about 5 mm² to 10 mm².

A particularly preferred form of electrode 108 for use in the present disclosure is an electrode array. Electrode arrays are capable of modulating the nerve in a spatially selective manner, as is known (see, e.g. [References 17, 18, 19]). Spatially-selective modulation of an eye-related sympathetic nerve (e.g. the ICN) is particularly useful for applying certain kinds of neural inhibition or block, such as a selective, differential or anodal block in selected nerve fibers. In particular, it is beneficial for selectively blocking A or C fibers.
[34] Saylam et al. Clinical Anatomy, 22:324-330.

The electrode arrays may be of the penetrating or non-penetrating type. A suitable electrode array may be an ICS-96 MultiPort planar array from Blackrock Microsystems. One possible configuration has 90 channels: 4×10 and 5×10 split planar arrays, with approximately 2000 mm² surface area, 1 mm shaft length, and 0.4 mm interelectrode spacing.

Exposed contacts 109 may be insulated by a non-conductive biocompatible material, which may be spaced transversely along the eye-related sympathetic nerve in use.

### Other Suitable Forms of Neural Interfacing Element and Signal

Optogenetics is a technique in which genetically-modified cells express photosensitive features, which can then be activated with light to modulate cell function. Many different optogenetic tools have been developed for inhibiting neural firing. A list of optogenetic tools to suppress neural activity is compiled in [Reference 35]. Acrylamine-azobenzene-quaternary ammonium (AAQ) is a photochromic ligand that blocks many types of K+ channels and in the cis configuration, the relief of K+ channel block inhibits firing [Reference 36]. Thus light can be used with genetic modification of target cells to achieve inhibition of neural activity.

The signal may use thermal energy, and the temperature of a nerve can be modified to inhibit the propagation of neural activity. For example, reference [Reference 37] discusses how cooling a nerve blocks signal conduction without an onset response, the block being both reversible and fast acting, with onsets of up to tens of seconds. Heating the nerve can also be used to block conduction, and is generally easier to implement in a small implantable or localised transducer or device, for example using infrared radiation from laser diode or a thermal heat source such as an electrically resistive element, which can be used to provide a fast, reversible, and spatially very localised heating effect (see for example reference [Reference 38]). Either heating, or cooling, or both could be conveniently provided in vivo using a Peltier element.
[35] Ritter L M et al., Epilepsia, 2014.
[36] Kramer et al., Optogenetic pharmacology for control of native neuronal signaling proteins, 2013; 16(7):816-23.
[37] Patherg et al. Blocking of impulse conduction in peripheral nerves by local cooling as a routine in animal experimentation. Journal of Neuroscience Methods 1984; 10:267-75.
[38] Duke et al. J Neural Eng. 2012 June; 9(3):036003. Spatial and temporal variability in response to hybrid electro-optical stimulation.

Where the signal applied to a nerve is a thermal signal, the signal can reduce the temperature of the nerve. In certain such embodiments the nerve is cooled to 14° C. or lower to partially inhibit neural activity, or to 6° C. or lower, for example 2° C., to fully inhibit neural activity. In such embodiments, it is preferably not to cause damage to the nerve. In certain alternative embodiments, the signal increases the temperature of the nerve. In certain embodiments, neural activity is inhibited by increasing the nerve's temperature by at least 5° C., for example by 5° C., 6° C., 7° C., 8° C., or more. In certain embodiments, signals can be used to heat and cool a nerve simultaneously at different locations on the nerve, or sequentially at the same or different location on the nerve.

The signal may comprise a mechanical signal. In certain embodiments, the mechanical signal is a pressure signal. In certain such embodiments, the neural interface is a transducer which causes a pressure of at least 250 mmHg to be applied to the nerve which inhibits neural activity. In certain alternative embodiments, the signal is an ultrasonic signal. In certain such embodiments, the ultrasonic signal has a frequency of 0.5-2.0 MHz, optionally 0.5-1.5 MHz, optionally 1.1 MHz. In certain embodiments, the ultrasonic signal has a density of 10-100 W/cm², for example 13.6 W/cm² or 93 W/cm².

Another mechanical form of signal for modulating neural activity uses ultrasound which may conveniently be implemented using external instead of implanted ultrasound transducers.

The signal may comprise an electromagnetic signal, such as an optical signal. Optical signals can conveniently be applied using a laser and/or a light emitting diode configured to apply the optical signal. In certain such embodiments, the optical signal (for example the laser signal) has an energy density from 500 mW/cm² to 900 W/cm². In certain alternative embodiments, the signal is a magnetic signal. In certain such embodiments, the magnetic signal is a biphasic signal with a frequency of 5-15 Hz, optionally 10 Hz. In certain such embodiments, the signal has a pulse duration of 1-1000 μs, for example 500 μs.

### Microprocessor

The implantable system 116, in particular the implantable device 106, may comprise a processor, for example microprocessor 113. Microprocessor 113 may be responsible for triggering the beginning and/or end of the signals delivered to the nerve (e.g., an eye-related sympathetic nerve) by the at least one neural interfacing element. Optionally, microprocessor 113 may also be responsible for generating and/or controlling the parameters of the signal.

Microprocessor 113 may be configured to operate in an open-loop fashion, wherein a pre-defined signal (e.g. as described above) is delivered to the nerve at a given periodicity for continuously) and for a given duration for indefinitely) with or without an external trigger, and without any control or feedback mechanism. Alternatively, microprocessor 113 may be configured to operate in a closed-loop fashion, wherein a signal is applied based on a control or feedback mechanism. As described elsewhere herein, the external trigger may be an external controller 101 operable by the operator to initiate delivery of a signal.

Microprocessor 113 of the implantable system 116, in particular of the implantable device 106, may be constructed so as to generate, in use, a preconfigured and/or operator-selectable signal that is independent of any input. Preferably, however, microprocessor 113 is responsive to an external signal, more preferably information (e.g. data) pertaining to one or more physiological parameters of the subject.

Microprocessor 113 may be triggered upon receipt of a signal generated by an operator, such as a physician or the subject in which the device 116 is implanted. To that end, the implantable system 116 may be part of a system which additionally comprises an external system 118 comprising a controller 101. An example of such a system is described below with reference to FIG. 12.

External system 118 of system 100 is external the implantable system 116 and external to the subject, and comprises controller 101. Controller 101 may be used for controlling and/or externally powering implantable system 116. To this end, controller 101 may comprise a powering unit 102 and/or a programming unit 103. The external system 118 may further comprise a power transmission antenna 104 and a data transmission antenna 105, as further described below.

The controller 101 and/or microprocessor 113 may be configured to apply any one or more of the above signals to the nerve intermittently or continuously. Intermittent application of a signal involves applying the signal in an (on-off)ₙ pattern, where n>1. For instance, the signal can be applied continuously for at least 5 days, optionally at least 7 days, before ceasing for a period (e.g. 1 day, 2 days, 3 days, 1 week, 2 weeks, 1 month), before being again applied continuously for at least 5 days, etc. Thus the signal is applied for a first time period, then stopped for a second time period, then reapplied for a third time period, then stopped for a fourth time period, etc. In such an embodiment, the first, second, third and fourth periods run sequentially and consecutively. The duration of the first, second, third and fourth time periods is independently selected. That is, the duration of each time period may be the same or different to any of the other time periods. In certain such embodiments, the duration of each of the first, second, third and fourth time periods may be any time from 1 second (s) to 10 days (d), 2 s to 7 d, 3 s to 4 d, 5 s to 24 hours (24 h), 30 s to 12 h, 1 min to 12 h, 5 min to 8 h, 5 min to 6 h, 10 min to 6 h, 10 min to 4 h, 30 min to 4 h, 1 h to 4 h. In certain embodiments, the duration of each of the first, second, third and fourth time periods is 5 s, 10 s, 30 s, 60 s, 2 min, 5 min, 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h, 24 h, 2 d, 3 d, 4 d, 5 d, 6 d, 7 d.

In certain embodiments, the signal is applied by controller 101 and/or microprocessor for a specific amount of time per day. In certain such embodiments, the signal is applied for 10 min, 20 min, 30 min, 40 min, 50 min, 60 min, 90 min, 2 h, 3 h, 4 h, 5 h, 6 h, 7 h, 8 h, 9 h, 10 h, 11 h, 12 h, 13 h, 14 h, 15 h, 16 h, 17 h, 18 h, 19 h, 20 h, 21 h, 22 h, 23 h per day. In certain such embodiments, the signal is applied continuously for the specified amount of time. In certain alternative such embodiments, the signal may be applied discontinuously across the day, provided the total time of application amounts to the specified time.

Continuous application may continue indefinitely, e.g. permanently. Alternatively, the continuous application may be for a minimum period, for example the signal may be continuously applied for at least 5 days, or at least 7 days.

Whether the signal applied to the nerve is controlled by controller 101, or whether the signal is continuously applied directly by microprocessor 113, although the signal might be a series of pulses, the gaps between those pulses do not mean the signal is not continuously applied.

In certain embodiments, the signal is applied only when the subject is in a specific state e.g. only when the subject is awake, only when the subject is asleep, prior to and/or after the ingestion of food, prior to and/or after the subject undertakes exercise, etc.

The various embodiments for timing for modulation of neural activity in the nerve can all be achieved using controller 101 in as device or system of the disclosure.

### Other Components of the System Including the Implantable Device

In addition to the aforementioned electrode 108 and microprocessor 113, the implantable system 116 may comprise one or more of the following components: implantable transceiver 110; physiological sensor 111; power source 112; memory 114; and physiological data processing module 115. Additionally or alternatively, the physiological sensor 111; memory 114; and physiological data processing module 115 may be part of a sub-system external to the implantable system. Optionally, the external sub-system may be capable of communicating with the implantable system, for example wirelessly via the implantable transceiver 110.

In some embodiments, one or more of the following components may preferably be contained in the implantable device 106: power source 112; memory 114; and a physiological data processing module 115.

The power source 112 may comprise a current source and/or a voltage source for providing the power for the signal delivered to an eye-related sympathetic nerve by the electrode 108. The power source 112 may also provide power for the other components of the implantable device 106 and/or implantable system 116, such as the microprocessor 113, memory 114, and implantable transceiver 110. The power source 112 may comprise a battery, the battery may be rechargeable.

It will be appreciated that the availability of power is limited in implantable devices, and the disclosure has been devised with this constraint in mind. The implantable device 106 and/or implantable system 116 may be powered by inductive powering or a rechargeable power source.

Memory 114 may store power data and data pertaining to the one or more physiological parameters from internal system 116. For instance, memory 114 may store data pertaining to one or more signals indicative of the one or more physiological parameters detected by physiological sensor 111, and/or the one or more corresponding physiological parameters determined via physiological data processing module 115. In addition or alternatively, memory 114 may store power data and data pertaining to the one or more physiological parameters from external system 118 via the implantable transceiver 110. To this end, the implantable transceiver 110 may form part of a communication subsystem of the system 100, as is further discussed below.

Physiological data processing module 115 is configured to process one or more signals indicative of one or more physiological parameters detected by the physiological sensor 111, to determine one or more corresponding physiological parameters. Physiological data processing module 115 may be configured for reducing the size of the data pertaining to the one or mom physiological parameters for storing in memory 114 and/or for transmitting to the external system via implantable transceiver 110. Implantable transceiver 110 may comprise a one or more antenna(e). The implantable transceiver 100 may use any suitable signaling process such as RF, wireless, infrared and so on, for transmitting signals outside of the body, for instance to system 100 of which the implantable system 116 is one part.

Alternatively or additionally, physiological data processing module 115 may be configured to process the signals indicative of the one or more physiological parameters and/or process the determined one or more physiological parameters to determine the evolution of the eye-related medical condition in the subject. In such case, the implantable system 116, in particular the implantable device 106, will include a capability of calibrating and tuning the signal parameters based on the one or more physiological parameters of the subject and the determined evolution of the eye-related medical condition in the subject, as is further discussed below.

The physiological data processing module 115 and the at least one physiological sensor 111 may form a physiological sensor subsystem, also known herein as a detector, either as part of the implantable system 116 put of the implantable device 106, or external to the implantable system.

Physiological sensor 111 comprises one or more sensors, each configured to detect a signal indicative of one of the one or more physiological parameters described above. For example, the physiological sensor 110 is configured for one or more of detecting electrodermal activity using an electrical sensor; detecting electroretinographic activity using an electrical sensor; detecting biomolecule concentration using electrical, RF or optical (visible, infrared) biochemical sensor; or a combination thereof.

The physiological parameters determined by the physiological data processing module 115 may be used to trigger the microprocessor 113 to deliver a signal of the kinds described above to an eye-related sympathetic nerve using the electrode 108. Upon receipt of the signal indicative of a physiological parameter received from physiological sensor 111, the physiological data processor 115 may determine the physiological parameter of the subject, and the evolution of the eye-related medical condition, by calculating in accordance with techniques known in the art.

The memory 114 may store physiological data pertaining to normal levels of the one or more physiological parameters. The data may be specific to the subject into which the implantable system 116 is implanted, and gleaned from various tests known in the art. Upon receipt of the signal indicative of a physiological parameter received from physiological sensor 111, or else periodically or upon demand from physiological sensor 111, the physiological data processor 115 may compare the physiological parameter determined from the signal received from physiological sensor 111 with the data pertaining to a normal level of the physiological parameter stored in the memory 114, and determine whether the received signals are indicative of insufficient or excessive of a particular physiological parameter, and thus indicative of the evolution of the eye-related medical condition in the subject.

The implantable system 116 and/or implantable device 106 may be configured such that if and when an insufficient or excessive level of a physiological parameter is determined by physiological data processor 115, the physiological data processor 115 triggers delivery of a signal to an eye-related sympathetic nerve by the neural interface (e.g. electrode 108), in the manner described elsewhere herein. For instance, if physiological parameter indicative of worsening of any of the physiological parameters and/or of the disease is determined, the physiological data processor 115 may trigger delivery of a signal which dampens secretion of the respective biochemical, as described elsewhere herein. Particular physiological parameters relevant to the present disclosure are described above. When one or more signals indicative of one or more of these physiological parameters are received by the physiological data processor 115, a signal may be applied to an eye-related sympathetic nerve via the electrode 108.

As an alternative to, or in addition to, the ability of the implantable system 116 and/or implantable device 106 to respond to physiological parameters of the subject, the microprocessor 113 may be triggered upon receipt of a signal generated by an operator (e.g. a physician or the subject in which the system 116 is implanted). To that end, the implantable system 116 may be part of a system 100 which comprises external system 118 and controller 101, as is further described below.

### System Including Implantable Device

With reference to FIG. 12, the implantable device 106 of the disclosure may be part of a system 110 that includes a number of subsystems, for example the implantable system 116 and the external system 118. The external system 118 may be used for powering and programming the implantable system 116 and/or the implantable device 106 through human skin and underlying tissues.

The external subsystem 118 may comprise, in addition to controller 101, one or more of: a powering unit 102, for wirelessly recharging the battery of power source 112 used to power the implantable device 106 and, a programming unit 103 configured to communicate with the implantable transceiver 110. The programming unit 103 and the implantable transceiver 110 may form a communication subsystem. In some embodiments, powering unit 102 is housed together with programming unit 103. In other embodiments, they can be housed in separate devices.

The external subsystem 118 may also comprise one or more of: power transmission antenna 104; and data transmission antenna 105. Power transmission antenna 104 may be configured for transmitting an electromagnetic field at a low frequency (e.g., from 30 kHz to 10 MHz). Data transmission antenna 105 may be configured to transmit data for programming or reprogramming the implantable device 106 and may be used in addition to the power transmission antenna 104 for transmitting an electromagnetic field at a high frequency (e.g., from 1 MHz to 10 GHz). The temperature in the skin will not increase by more than 2 degrees Celsius above the surrounding tissue during the operation of the power transmission antenna 104. The at least one antennae of the implantable transceiver 110 may be configured to receive power from the external electromagnetic field generated by power transmission antenna 104, which may be used to charge the rechargeable battery of power source 112.

The power transmission antenna 104, data transmission antenna 105, and the at least one antennae of implantable transceiver 110 have certain characteristics such a resonant frequency and a quality factor (Q). One implementation of the antenna(e) is a coil of wire with or without a ferrite core forming an inductor with a defined inductance. This inductor may be coupled with a resonating capacitor and a resistive loss to form the resonant circuit. The frequency is set to match that of the electromagnetic field generated by the power transmission antenna 105. A second antenna of the at least one antennae of implantable transceiver 110 can be used in implantable system 116 for data reception and transmission from/to the external system 118. If more than one antenna is used in the implantable system 116, these antennae are rotated 30 degrees from one another to achieve a better degree of power transfer efficiency during slight misalignment with the with power transmission antenna 104.

External system 118 may comprise one or more external body-worn physiological sensors 121 (not shown) to detect signals indicative of one or more physiological parameters. The signals may be transmitted to the implantable system 116 via the at least one antennae of implantable transceiver 110. Alternatively or additionally, the signals may be transmitted to the external system 116 and then to the implantable system 116 via the at least one antennae of implantable transceiver 110. As with signals indicative of one or more physiological parameters detected by the implanted physiological sensor 111, the signals indicative of one or more physiological parameters detected by the external sensor 121 may be processed by the physiological data processing module 115 to determine the one or more physiological parameters and/or stored in memory 114 to operate the implantable system 116 in a closed-loop fashion. The physiological parameters of the subject determined via signals received from the external sensor 121 may be used in addition to alternatively to the physiological parameters determined via signals received from the implanted physiological sensor 111.

For example, in a particular embodiment a detector external to the implantable device may include an optical detector including a camera capable of imaging the eye and determining changes in physiological parameters, in particular the physiological parameters described above. As explained above, in response to the determination of one or more of these physiological parameters, the detector may trigger delivery of signal to an eye-related sympathetic nerve by the electrode 108, or may modify the parameters of the signal being delivered or a signal to be delivered to an eye-related sympathetic time by the electrode 108 in the future.

The system 100 may include a safety protection feature that discontinues the electrical modulation of an eye-related sympathetic nerve in the following exemplary events: abnormal operation of the implantable system 116 (e.g. overvoltage); abnormal readout from an implanted physiological sensor 111 (e.g. temperature increase of more than 2 degrees Celsius or excessively high or low electrical impedance at the electrode-tissue interface); abnormal readout from an external body-worn physiological sensor 121 (not shown); or abnormal response to inhibition/blocking detected by an operator (e.g. a physician or the subject). The safety precaution feature may be implemented via controller 101 and communicated to the implantable system 116, or internally within the implantable system 116.

The external system 118 may comprise an actuator 120 (not shown) which, upon being pressed by an operator (e.g. a physician or the subject), will deliver a signal, via controller 101 and the respective communication subsystem, to trigger the microprocessor 113 of the implantable system 116 to deliver a signal to the nerve by the electrode 108.

System 100 of the disclosure, including the external system 118, but in particular implantable system 116, is preferably made from, or coated with, a biostable and biocompatible material. This means that the device or system is both protected from damage due to exposure to the body's tissues and also minimizes the risk that the device or system elicits an unfavorable reaction by the host (which could ultimately lead to rejection). The material used to make or coat the device or system should ideally resist the formation of biofilms. Suitable materials include, but are not limited to, poly (p-xylylene) polymers (known as Parylenes) and polytetrafluoroethylene.

The implantable device 116 of the disclosure will generally weigh less than 50 g.

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the disclosure.

The term “about” in relation to a numerical value x is optional and means, for example, x±10%.

Unless otherwise indicated each embodiment as described herein may be combined with another embodiment as described herein.

### MODES FOR CARRYING OUT THE DISCLOSURE

### Experimental Study I

The aim of the first experimental study was to test the validity of neural modulation. In particular, to demonstrate whether the effects of laser photocoagulation can be improved or worsened by modulating sympathetic activity, either through ICN denervation or by local β-adrenoreceptor (β-AR) agonist or antagonist administration. Endpoints include the extent of choroidal neovascularization (CNV) and levels of angiogenic growth factors.

### Background

The laser photocoagulation model is the most widely accepted animal model of wet AMD [Reference 39]. The model works by burning Bruch's membrane with a laser, which causes growth of new blood vessels from the choroid into the subretinal space [40]. This growth is accompanied by upregulation of VEGF [References 41; 42] and TNF-α [References 43; 44]. Maximal changes are observed 1-2 weeks following laser injury. Two
[39] Pennesi, et al., Molecular Aspects of Medicine, 2012; 33:487-509.
[40] Lambert et al., Nature Protocols, 2013, 8:2197-2211.
[41] Yi et al., Graefe's Archive for Clinical and Experimental Ophthalmology, 1997; 235:313-319.
[42] Wada et. al., Current Eye Research, 1999; 18:203-213.
[43] Shi et. al., Experimental Eye Research, 2006; 83:1325-1334.
[44] Jasielska et al., Investigative Ophthalmology & Visual Science, 2010; 51:3874-3883.
   methods are typically used to evaluate the extent of laser-induced CNV. In the first, fluorescein angiography (FA) is performed to assess leakage from newly formed vessels [Reference 45]. In the second, CNV volume is quantified by fluorescently labeling endothelial cells in choroidal flatmounts, followed by 3D reconstruction and volume analysis with confocal microscopy [Reference 46].

### Methods

In this experimental study, how increasing or decreasing ocular sympathetic activity affected laser-induced CNV progression were measured. Female Brown Norway rats (n=40), aged ˜P100, underwent laser photocoagulation with a green diode laser. Animals were divided into four treatment groups (n=10 per group):
1. Control group—Following laser injury, animals received daily eye drops of artificial tears (1.4% polyvinyl alcohol; Akorn, Lake Forest, Ill.) for 14 days.
2. β-AR agonist group—Following laser injury, animals received daily eye drops of 50 mM isoproterenol [Reference 47] dissolved in artificial tears for 14 days.
3. β-AR antagonist group—Following laser injury, animals received daily eye drops of 2% propranolol [Reference 48] dissolved in artificial tears for 14 days.
4. ICNx group—Animals underwent bilateral ICN transection 6 weeks prior to laser photocoagulation. Following laser injury, animals received daily eye drops of artificial tears for 14 days.

Half of the rats in each group received 4 laser burns per eye, while the other half received 12 burns in a single eye. (Twelve burns is considered a blinding procedure, so only one eye could be treated according to animal care guidelines.) All animals were euthanized 14 days after laser injury. Animals with 4 burns per eye were used for evaluating CNV development, while animals with 12 burns were used for evaluating VEGF protein expression. CNV development was quantified with FA scoring (performed prior to sacrifice) and lesion volume analysis using 3D confocal reconstruction [Reference 49]. Protein
[45] Takehana et al., Investigative Ophthalmology & Visual Science, 1999; 40:459-466.
[46] Campos et al., Investigative Ophthalmology & Visual Science, 2006; 47:5163-5170.
[47] Jiang et al., Experimental Eye Research, 2010; 91:171-179.
[48] Dal Monte et al., Experimental Eye Research, 2013; 111:27-35.
[49] He et. al., Investigative Ophthalmology & Visual Science, 2005; 46:4772-4779.
   expression was measured with ELISA [Reference 50]. Investigators who performed data analysis were blinded to the treatment group.

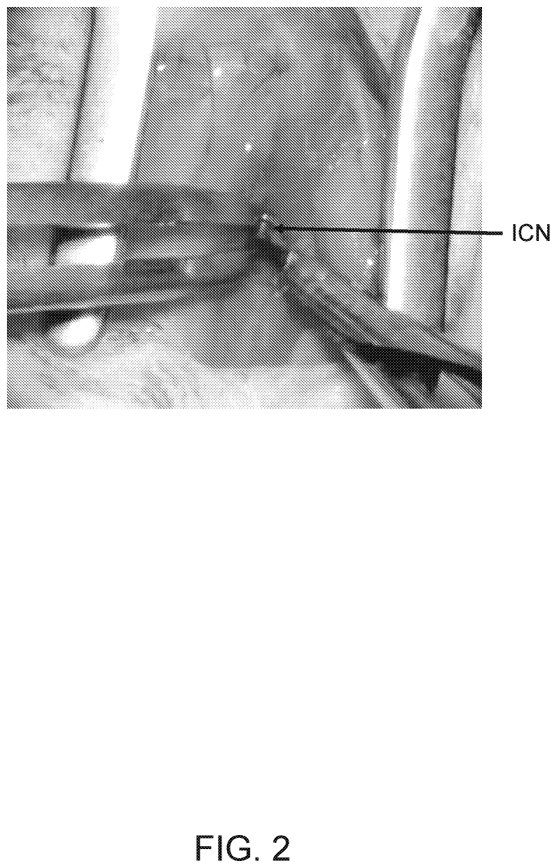

For ICN transection, rats were anesthetized with ketamine/xylazine placed in the supine position in order to expose ventral structures of the neck. Upper limbs were extended, providing better exposition of the surgical area. A vertical incision was made in the middle of the neck. The incision began 2 cm below the intermandibular region in the presternal region. The skin was retracted, and tissue underneath was dissected by blunt dissection, including superficial cervical fascia with mandibular glands. Neck muscles were exposed (sternohyoid, ornohyoid, sternomastoid, and posterior belly of the digastric muscles), and the carotid triangle was located between the muscles. Within the triangle, the carotid bifurcation was identified and separated into its structures (external and internal carotid arteries). The occipital artery quad hypoglossal nerve were clearly observed. The SCG was identified below those structures, and the internal and external carotid nerves were exposed. The ICN was fully transected distal to the SCG, beneath/adjacent to the hypoglossal nerve (FIG. 2A). Following transection, the skin incision was closed with a non-absorbable suture (nylon 6-0), and antibiotic ointment was applied. To verify successful surgery, eyelid and eyeball position were evaluated over the next 3 days. Ptosis was generally observed within 4-12 hrs after surgery, followed by exophthalmos between 12-24 hrs. Permanent ptosis ensued ˜24 hrs after ICN transection. Animals without apparent ptosis were euthanized.

For laser photocoagulation, rats were anesthetized ketamine/xylazine, and eye drops were instilled (1% tropicamide and 2.5% phenylephrine HCl) to induce full dilation. Rats were placed in the prone position in front of a slit lamp attached to a 532-nm OcuLight GL green diode laser (IRIDEX, Toronto, ON). Laser settings were: 150-160 mW power, 50 ms duration, and 75 μm diameter. The retina and optic nerve were visualized using a glass coverslip and lubricant gel over the eyes. In the group of animals that received 4 laser burns per eye, one burn was made per quadrant. In the group that received 12 burns in a single eye, one burn was made per clock hour. Care was taken to avoid the retinal vessels. Rupture of Burch's membrane was confirmed by the presence of a retinal bubble. Animals whose laser burns produced massive subretinal bleeding were euthanized immediately.
[50] Chan et al., Laboratory Investigation, 2005; 85:721-733.

Eye drops were applied between 9:00 and 11:00 am each day by instilling 2 drops (˜40 μL each) per eye. Isoproterenol is subject to oxidation, especially at pH levels ≥6.5 [Reference 51]. However, the pH of the eye drops could not be adjusted below 6.5, since that would irritate the eyes. In order to mitigate chemical degradation, isoproterenol and propranolol drops were freshly prepared once per week in plastic vials and stored at 4° C. UV spectra of the isoproterenol drops were measured over time to assess drug stability.

For performing fluorescein angiography (FA), animals were anesthetized with ketamine/xylazine followed by dilation eye drops (1% tropicamide and 2.5% phenylephrine HCl) and 0.01 mL intraperitoneal injection of 10% sodium fluorescein dye. Sequential posterior pole images were taken using a RetCam 3 retinal camera (Clarity Medical Systems, Pleasanton, Calif.) with an 80° lens. The intensity of fluorescein staining in late-phase FAs was scored according to an established grading scale. Lesions were given a score of 0 (no staining), 1 (slightly stained), 2 (moderately stained), or 3 (strongly stained). The scores of all lesions within each treatment group were averaged. Lesions that scored a 0 were excluded from analysis, since those lesions likely represented laser impacts that did not result in CNV [Reference 40].

To perform lesion volume analysis, animals (n=5 per group) were euthanized at least 4-5 hours after FA imagine, in order to allow the fluorescein enough time to clear from the circulatory system. The cornea, lens, and retina were removed from the eyes. The sclera-choroid complex was fixed overnight in 4% formalin at 4° C. Tissue was washed the next day and permeabilized with 0.5% Triton-X for 4 hrs. The eye cups were blocked with 1% BSA for 2 hours and placed in 1:50 fluorescein-labeled GSL I isolectin B4 (endothelial cell marker; Vector Laboratories, Burlingame, Calif.) at 4° C. overnight. Samples were washed and mounted on slides with mounting media (VECTASHIELD; Vector Laboratories), while making incisions in the eye cups to flatten them.

Flatmounts were visualized using the 10× objective of an UltraVIEW spinning disk confocal microscope (PerkinElmer, Waltham, Mass.). The image stacks were generated in the z-plane, with the microscope set to excite at 488 nm and to detect at 505-530 nm. Images were processed using the microscope's software, by closely circumscribing and digitally
[51] Leach et al., American Journal of Hospital Pharmacy, 1977; 34:709-712.
   extracting the fluorescent lesion areas throughout the entire image stack. The extracted lesion was processed through the topography software to generate a digital topographic image representation of the lesion and an image volume. The topographic analysis program determines and displays the objects' surface contours by detecting fluorescent signal from the top of the image stack and then measures everything under the surface to yield a final volume, which reflects the CNV fluorescence volume.

To determine the angiogenic growth factor expression, animals (n=5 per group) were euthanized 14 days after laser photocoagulation. Eyes that received 12 burns were enucleated. Posterior poles were isolated from each eye and pooled within each of the 4 treatment groups. Tissues were homogenized in 250 μL of buffer (80 mM Tris-HCl, 4 mM MgCl₂, 0.5 mM phenylmethylsulfonyl fluoride) containing mixed protease inhibitors (Roche, Basel, Switzerland) for protein extraction. Protein homogenates were centrifuged at 14,000 g for 10 min to remove tissue debris. Total protein concentration was determined by a Bio-Rad protein assay (Bio-Rad Laboratories, Hercules, Calif.). VEGF protein expression in the posterior poles was assessed with 75 μg total protein per sample in triplicate with a VEGF ELISA kit (R&D Systems, Minneapolis, Minn.). The detection range of this assay is 3-500 pg/mL.

### Results and Discussion

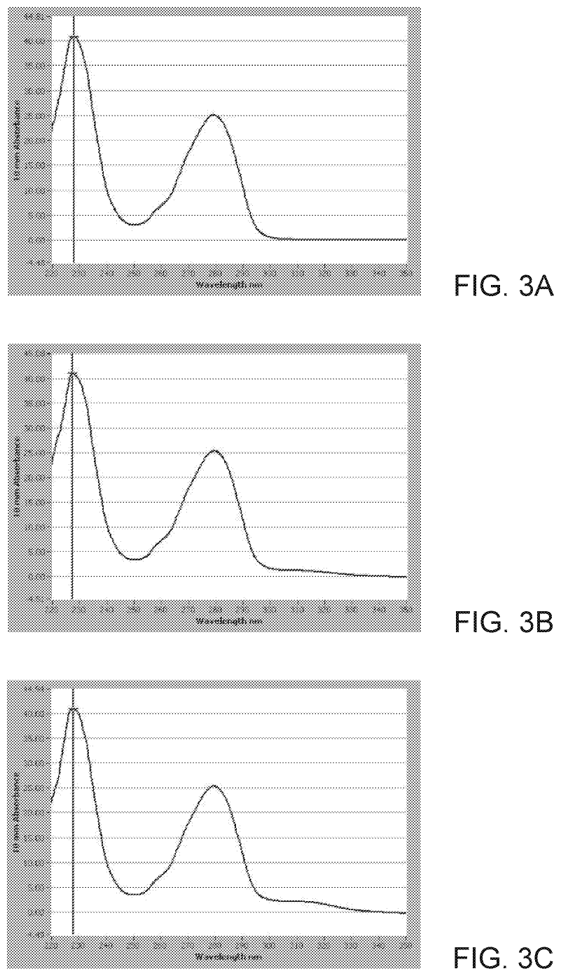

UV spectra of isoproterenol eye drops (50 mM in artificial tears) was measured over a 2-wk period to confirm lack of oxidation degradation. Spectra remained consistent over this time period (see FIGS. 3A, 3B and 3C) and were identical to published spectra [Reference 52]. A fresh batch of eye drops was mixed at least once per week for use in animal experiments.

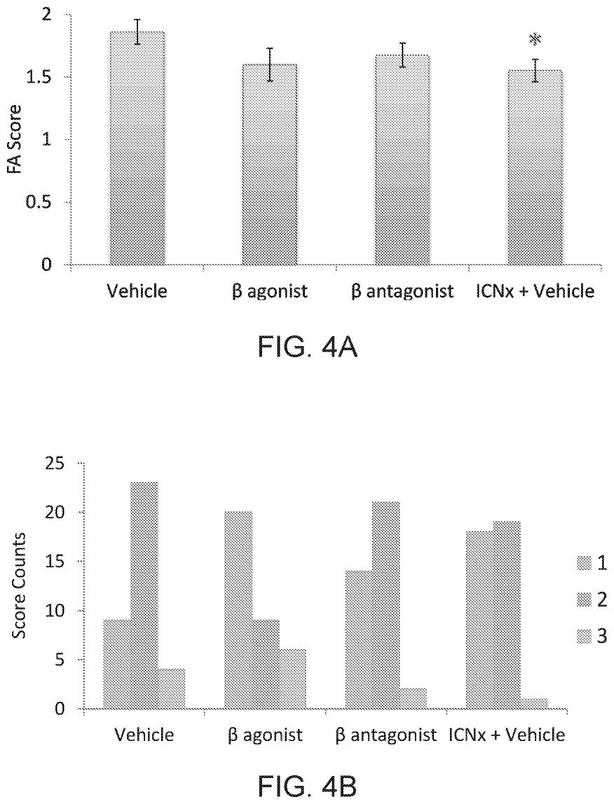

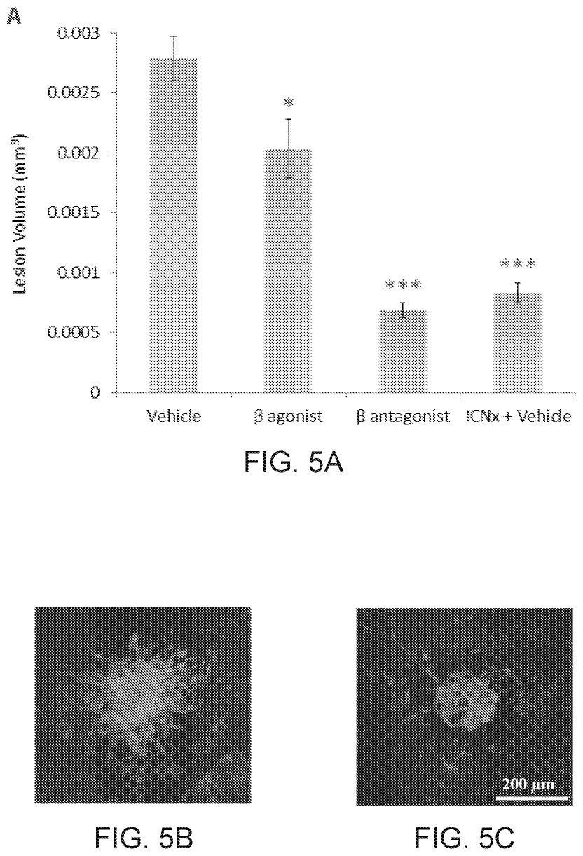

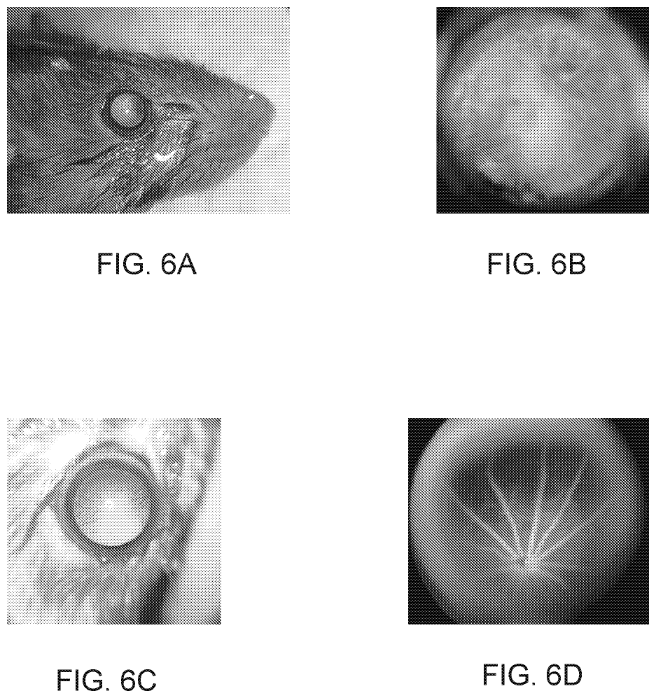

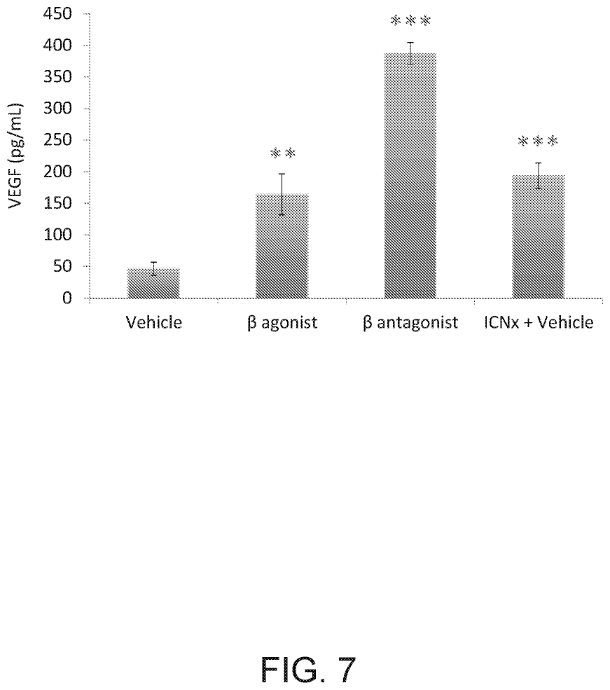

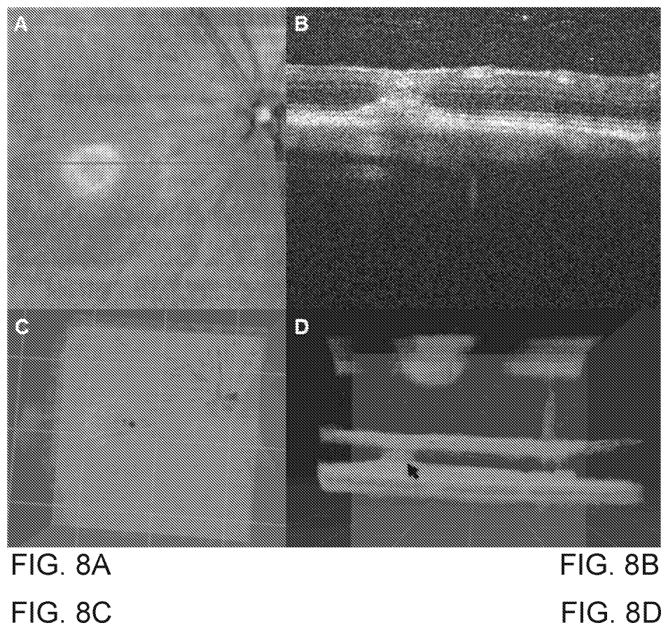

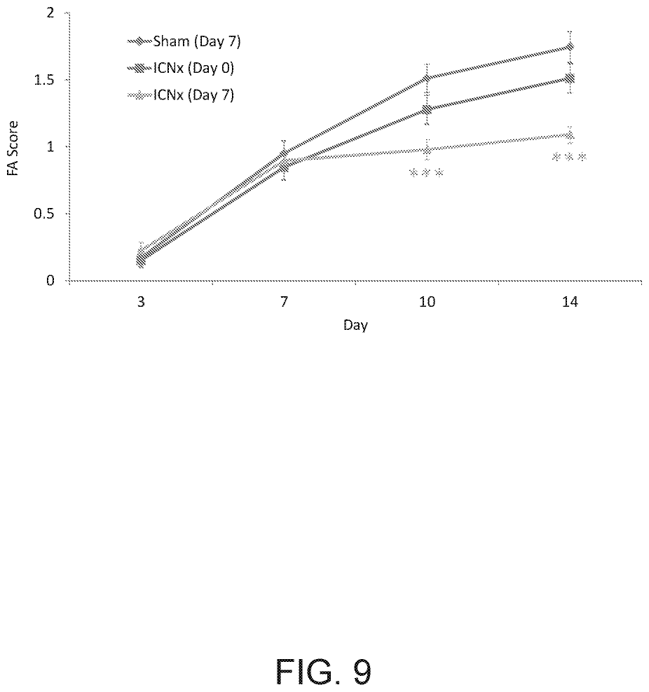

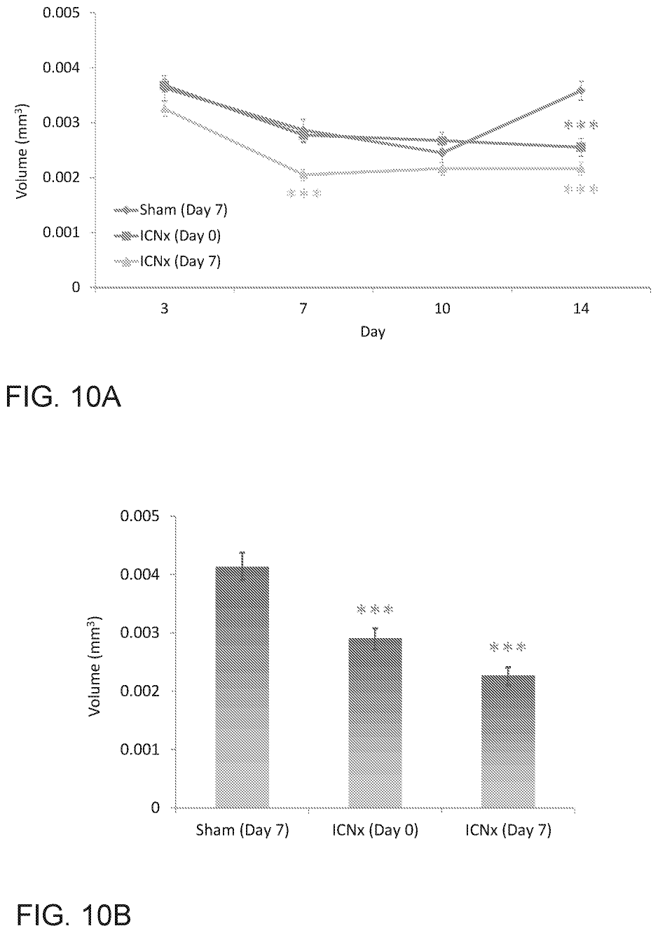

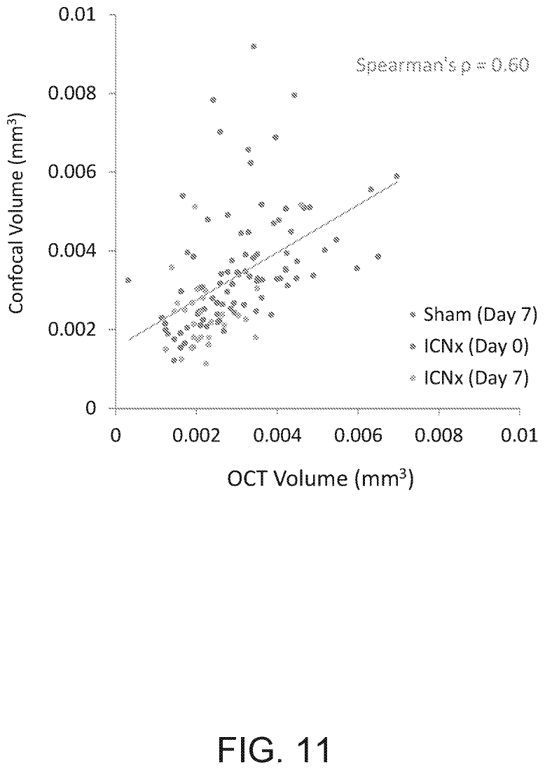

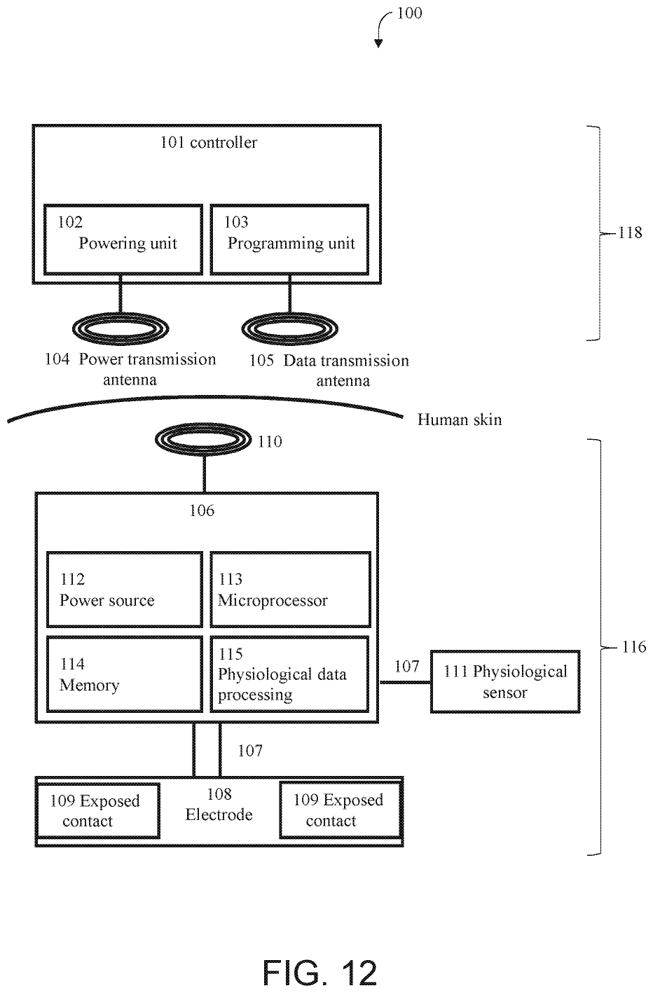

Average FA scores for all groups fell between 1.5 and 2.0, indicating moderate staining. Lesions in rats receiving daily β-AR eye drops (propranolol or isoproterenol) were slightly less leaky than lesions in the vehicle group; however, scores were not significantly different among, these groups (P>0.05). In rats that underwent ICN transection 6 weeks prior
[52] Siva et al., Physics and Chemistry of Liquids, 2012; 50:434-452.
   to laser treatment, FA scores were 17% lower than scores in the control group (P=0.02, unpaired t-test). The results are summarized in FIG. 4A and FIG. 4B.

Analysis of lesion volumes postmortem revealed smaller lesions in the 3 treatment groups than in the control group, implicating a clear role for the β-AR system in regulating CNV. Blocking β-AR activity with propranolol or through ICN denervation led to reductions in lesion volume by 75% and 70%, respectively (P<0.001, unpaired t-tests; see FIG. 5A). In agreement with the FA data (see FIG. 4A and FIG. 4B), these results suggest that blocking ocular sympathetic, activity is anti-angiogenic. Unexpectedly, application of the β-AR agonist isoproterenol also led to a reduction in lesion volume versus the control group (27%, P<0.05). This could arise from β-AR desensitization or downregulation caused by prolonged application of isoproterenol, as reported by others [References 53; 54; 55; 56].

Though no studies have investigated the effect of β-AR agonists on the progression of laser-induced CNV, others have reported that systemic or intraocular delivery of the β-AR antagonist propranolol causes a reduction in CNV lesion size. Both of these studies were performed in mouse. In [Reference 57], Lavine et al. measured lesion areas (as opposed to volumes) 14 days after laser photocoagulation and found that daily intraperitoneal injection of propranolol (20 mg/kg) led to a 50% reduction in lesion size. In [Reference 58], Nourinia et al. measured lesion areas 28 days after laser photocoagulation and found that a single intravitreal injection of propranolol (0.3 μg) at the time of laser application led to a 79% reduction in lesion size, similar to what the inventors observed.

An unexpected side effect of propranolol eye drop administration was development of corneal neovascularization (see FIGS. 6A-6D). This was a surprising finding, given that propranolol led to suppression of laser-induced CNV in the same animals (see FIG. 5A). Thus, it appears that β-AR blocking can have opposing effects in different regions of the eye.
[53] Dal Monte et al., Investigative Ophthalmology & Visual Science, 2012; 53:2181-2192.
[54] Gonzalez-Brito et al., Life Sciences, 1988; 43:707-714.
[55] Gambarana et al., Brain Research, 1991; 555:141-148.
[56] Brouri et al., European Journal of Pharmacology, 2002; 456:69-75.
[57] Lavine et al., JAMA Ophthalmology, 2013; 131:376-382.
[58] Nourinia et al., Investigative Ophthalmology & Visual Science, 2015; 56:8228-8235.
   To our knowledge, corneal neovascularization arising front β-AR blocking has never been reported.

ELISA performed 14 days after laser photocoagulation indicated elevated choroidal VEGF levels in the 3 treatment groups versus the control group (see FIG. 7). VEGF levels in the control group were relatively low, suggesting a return to baseline in this group. Protein levels in the treatment groups were significantly higher (P<0.01, unpaired t-tests), which is unexpected since lesions were smaller and slightly less leaky in these groups (see FIG. 4 and FIG. 5). With higher VEGF levels, larger lesions would be expected, i.e. the opposite of what was observed. There are a few possible explanations for why VEGF levels were elevated in the treatment groups: First, VEGF was measured 14 days after laser photocoagulation, which may not have been the appropriate time point (for example, systemic VEGF levels in mouse peak 7 days after laser photocoagulation and return to baseline after 14 days [59]). VEGF levels are affected by several factors including inflammation, ischemia, and hypoxia. It is possible that treatment (eye drop administration or ICNx surgery) caused these side effects. In patients with wet AMD, anti-VEGF therapy reduces leakiness but not CNV lesion size, which could explain why VEGF levels in our experiments did not correlate with lesion volume. Finally, VEGF were highest in the group that received propranolol eye drops. Animals in this group exhibited corneal neovascularization (see FIG. 6), which may have contributed to the elevated VEGF levels.

### Experimental Study 2

The aim of this follow-on study was to further validate the effects of ICN denervation. In particular, to demonstrate that ICN denervation, when performed after laser photocoagulation, can mitigate CNV. Endpoints include the extent of CNV and levels of angiogenic growth factors.

This follow-on study was conducted to further investigate the effects of ICN denervation in the rat laser-induced CNV model. Results from the initial study revealed that blocking ocular sympathetic activity in this model mitigated progression of laser-induced CNV (see FIG. 4 and FIG. 5). However, ICN denervation was performed 6 weeks prior to laser photocoagulation. An electromodulation therapy for wet AMD would not commence
[59] Kase et al., Blood, 2010; 115:3398-3406.
   until after a patient presents with CNV; therefore, a better animal model of the clinical situation would be to perform ICN denervation after laser photocoagulation. That was the purpose of the follow-on study.

### Methods

A total of 18 rats (male Brown Norway, ˜P100) underwent laser photocoagulation with a green diode laser (150-160 mW, 50 ms, 75 μm). (Male animals were used in order to avoid possible confounding effects from the menstrual cycle on VEGF levels.) Animals received 4 burns per eye and were euthanized 14 days later. Rats were divided into three experimental groups (n=6 per group):
1. Sham (Day 7) group—Animals underwent bilateral sham surgery 7 days after laser therapy. The ICNs were exposed but not transected.
2. ICNx (Day 0) group—Animals underwent bilateral ICN transection immediately after laser therapy.
3. ICNx (Day 7) group—Animals underwent bilateral ICN transection 7 days after laser therapy.

The following analyses, summarized in Table 2, were performed to evaluate CNV progression over time. Investigators who performed the analyses were blinded to the treatment group.

TABLE 2
Analyses performed to track CNV progression during the follow-on
study. The minus and plus signs refer to procedures performed
before and after treatment, respectively.

| Analysis | Time point(s) | Measurement method |
|---|---|---|
| Lesion volume and | Days 0⁺, 3, 7⁻, 10, 14 | SD-OCT |
| edema (in vivo) | | |
| CNV leakiness | Days 3, 7⁻, 10,14 | FA |
| Lesion volume (ex vivo) | After sacrifice | 3D confocal |
| | | reconstruction |

Spectral-domain optical coherence tomography (SD-OCT) was used to monitor CNV progression in vivo (see FIG. 8). OCT imaging was performed with an Envisu Bioptigen system (Leica Microsystems, Wetzlar, Germany) to assess in vivo progression of laser lesions after photocoagulation. Each lesion was imaged using 100 horizontal raster scans spaced 16 μm apart, over an area of 1.6×1.6 mm. A stereological method (three-dimensional interpretation of two-dimensional cross sections) was used to reconstruct the OCT images in 3D and calculate lesion size. For this, the “Volumest” (volume estimation) plug-in [Reference 60] of ImageJ was used. Each image section passing through the lesion was delineated and measured by hand. In each image, the lesion was identified as the subretinal hyperreflective material above the retinal pigment epithelium.

FA was performed to assess leakage from newly formed vessels according to an established grading scale (see Methods from the initial study).

Following euthanasia, CNV volume was quantified by fluorescently labeling endothelial cells in choroidal flatmounts, followed by 3D reconstruction and volume analysis with confocal microscopy (see Methods from the initial study). To correlate OCT (in vivo) and confocal (ex vivo) lesion volumes, each lesion was identified according to its position relative to the optic nerve: superotemporal, inferotemporal, superonasal, and inferonasal.

### Results and Discussion

Consistent with results from the initial study, data from the follow-on study indicated that blocking ocular sympathetic activity through ICN transection mitigated laser-induced CNV progression. FIG. 9 shows the FA scores in each experimental group at 3, 7, 10, and 14 days after laser photocoagulation. Scores in all groups were statistically similar on days 3 and 7, with the average score increasing from ˜0.2 to ˜0.9 over this time period. Scores in each group increased again on day 10 and on day 14, indicating steady CNV development throughout the 2-week monitoring period. On days 10 and 14, FA scores in both ICNx groups were lower than those of the control group, with the ICNx (Day 7) group exhibiting the lowest average scores. The average FA score in the ICNx (Day 7) group increased from ˜0.9 to ˜1.1 between days 7 and 14, signifying limited CNV progression over this time period.

FIG. 10 summarizes the results from the OCT and confocal lesion volume measurements. Lesions in all three groups shrank between days 3 and 7 after laser
[60] Merzin M., “Applying stereological method in radiology. Volume measurement.” thesis, University of Tartu, Estonia, 2008.
   photocoagulation, due to resolution of edema during this time period. Between days 7 and 10, lesion sizes remained relatively stable. Average lesion sizes in all groups were statistically similar through day 10, with just one exception (see FIG. 10A). By day 14, however, lesions in the sham group had grown, while lesion sizes in the two IC groups remained stable.

Both OCT and confocal imaging revealed significantly smaller lesion sizes in the ICNx groups versus the sham group on day 14 (P<0.001, unpaired t-tests). According to the confocal measurements, average lesion volume in the ICNx (Day 0) and ICNx (Day 7) groups was 30% and 45% smaller than that of the Sham (Day 7) group, respectively. For comparison, a mouse study with the FDA-approved anti-VEGF agent aflibercept (Eylea; VEGF-TRAPR1R2) found that a single intravitreal injection of the drug led to a ˜30% reduction in lesion size after 14 days [Reference 61].

Both the lesion volume and FA results indicate that denervating the ocular sympathetic supply mitigates progression of laser-induced CNV. Unexpectedly, it was found that denervating 7 days after laser treatment was more effective than denervating immediately after (see FIGS. 9 and 10). This finding may arise from use of relatively small sample sizes (n=6 animals per group). In support of this theory, it would be expected that 7 days after laser photocoagulation (before surgery), lesions in the ICNx (Day 7) group would be similar in size to lesions in the Sham (Day 7) group. However, as shown in FIG. 10A, ICNx (Day 7) group lesions were smaller than Sham (Day 7) group lesions at day 7. Thus, it may be worth repeating the follow-on study experiments with larger sample sizes.

Use of SD-OCT for monitoring laser-induced CNV lesion volume in vivo is a relatively new technique. Using this method in mice, there was shown to be good correlation between lesion sizes measured with OCT and with confocal at days 5 and 7 post-laser. However, measurements on days 14 and 28 did not show a statistically significant correlation. Furthermore, direct comparisons between volumes measured with OCT and those measured with confocal were not reported [Reference 62]. As shown in FIG. 11, the
[61] Saishin et al., Journal of Cellular Physiology, 2003; 195:241-248.
[62] Giani et al., Investigative Ophthalmology & Visual Science, 2011; 52:3880-3887.
   inventors were the first to demonstrate that lesion volumes measured by OCT are similar in size to those measured by the gold standard of confocal.

### Conclusions

Results from both the first and second experimental studies indicate that blocking ocular sympathetic activity inhibits CNV. These findings were made in the laser-induced CNV model, which has become the gold standard for preclinical testing of all current treatment modalities used to date for subretinal neovascularization [Reference 63]. Even when sympathetic denervation was performed 1 week after laser photocoagulation, mitigation of CNV progression was still observed. These results suggest that electrical modulation (e.g. inhibition) of ICN neural activity could be an effective strategy for treating eye disorders, e.g. eye disorders that are associated with subretinal neovascularization, such as wet AMD, or ocular neovascular diseases caused by injury to the eye.
[63] Pennesi et al., Molecular Aspects of Medicine, 2012; 33:487-509.

1.-32. (canceled)

33. A device or system comprising at least one neural interfacing electrode placed on, in, or around an eye-related sympathetic nerve, and a voltage or current source configured to generate an electrical signal to be applied to the eye-related sympathetic nerve via the at least one neural interfacing electrode wherein the electrical signal reversibly inhibits neural activity of the eye-related sympathetic nerve to produce a change in a physiological parameter in a subject, wherein the physiological parameter is one or more of the group consisting of: a level of an angiogenic growth factor in the eye, ocular blood flow, blood pressure, blood oxygenation, an extent of vision impairment, a level of an immune response modulator in the eye, an extent of blood vessel leakage in the eye, an amount or size of drusen deposits in the eye, an extent of macular edema, an extent of retinal cell death, a level of an oxidative stress marker, and a level of a peroxynitrite marker.

34. The device or system of claim 33, wherein the eye-related sympathetic nerve is modulated at an internal carotid nerve (ICN).

35. The device or system of claim 33, wherein the eye-related sympathetic nerve is modulated unilaterally or bilaterally.

36. The device or system of claim 33, wherein the electrical signal comprises a charge-balanced DC signal or a charge-balanced AC signal.

37. The device or system of claim 33, wherein the change in the physiological parameter is one or more of the group consisting of: a decrease in the level of a pro-angiogenic growth factor in the eye, an increase in the level of an anti-angiogenic growth factor in the eye, a decrease in choroidal neovascularization, a decrease in macular edema, a decrease in the amount of drusen deposits or size thereof, an improvement in central vision, a decrease in retinal cell death, an increase in blood oxygenation, a decrease in the level of an oxidative stress marker, and a decrease in the level of a peroxynitrite marker.

38. The device or system of claim 33, wherein the electrical signal has a frequency between 0.5 kHz and 100 kHz.

39. The device or system of claim 33, comprising a detector for detecting one or more signals indicative of one or more physiological parameters; determining from the one or more signals one or more physiological parameters; determining the one or more physiological parameters indicative of worsening of the physiological parameter; and causing the signal to be applied to the eye-related sympathetic nerve via the at least one electrode.

40. The device or system of claim 39, further comprising a memory for storing data pertaining to physiological parameters in a healthy subject, wherein determining the one or more physiological parameters indicative of worsening of the physiological parameter comprises comparing the one or more physiological parameters with the data.

41. The device or system of claim 33, comprising a communication subsystem for receiving a control signal from a controller and, upon detection of said one or more control signals, cause the electrical signal to be applied to the eye-related sympathetic nerve via the at least one electrode.

42. A method of reversibly inhibiting neural activity in the internal carotid nerve (ICN) comprising (i) implanting in a subject a device or system comprising at least one neural interfacing electrode placed on, in, or around the ICN, and a voltage or current source configured to generate an electrical signal to be applied to the ICN via the at least one neural interfacing electrode wherein the electrical signal reversibly inhibits neural activity of the ICN to produce a change in a physiological parameter in a subject, wherein the physiological parameter is one or more of the group consisting of: a level of an angiogenic growth factor in the eye, neovascularization ocular blood flow, blood pressure, blood oxygenation, an extent of vision impairment, a level of an immune response modulator in the eye, an extent of blood vessel leakage in the eye, an amount or size of drusen deposits in the eye, an extent of macular edema, an extent of retinal cell death, a level of an oxidative stress marker, and a level of a peroxynitrite marker; and (ii) applying an electrical signal to the ICN to inhibit the neural activity of the ICN

43. The method of claim 42, wherein the method mitigates choroidal neovascularization (CNV).

44. The method of claim 42, wherein the method treats an eye disorder associated with ocular neovascularization.

45. The method of claim 42, wherein the change in the physiological parameter is one or more of the group consisting of: a decrease in the level of a pro-angiogenic growth factor in the eye, an increase in the level of an anti-angiogenic growth factor in the eye, a decrease in choroidal neovascularization, a decrease in macular edema, a decrease in the amount of drusen deposits or size thereof, an improvement in central vision, a decrease in retinal cell death, an increase in blood oxygenation, a decrease in the level of an oxidative stress marker, and a decrease in the level of a peroxynitrite marker.

46. The method of claim 42, wherein the electrical signal has a frequency between 0.5 kHz and 100 kHz.

47. The method of claim 42, wherein the method is for treating wet AMD or an ocular neovascular disease caused by injury to the eye.

48. A method of reversibly inhibiting neural activity in an eye-related sympathetic nerve, comprising: (i) implanting in a subject a device or system of claim 33 and (ii) positioning the neural interfacing element in signaling contact with the eye-related sympathetic nerve.

49. The method of claim 48, wherein the method is for treating an eye disorder, such as an ocular neovascular disease.

50. The method of claim 49, wherein the method is for treating wet AMD or an ocular neovascular disease caused by injury to the eye.

51. A method for treating an eye disorder, comprising applying an electrical signal to an eye-related sympathetic nerve via at least one neural interfacing electrode, wherein the signal reversibly inhibits neural activity of the eye-related sympathetic nerve to produce a change in a physiological parameter in a subject, wherein the at least one neural interfacing electrode is suitable for placement on, in, or around the eye-related sympathetic nerve, wherein the physiological parameter is one or more of the group consisting of: a level of an angiogenic growth factor in the eye, neovascularization ocular blood flow, blood pressure, blood oxygenation, the extent of vision impairment, a level of an immune response modulator in the eye, an extent of blood vessel leakage in the eye, an amount or size of drusen deposits in the eye, an extent of macular edema, an extent of retinal cell death, a level of an oxidative stress marker, and a level of a peroxynitrite marker.

52. The method of claim 51, wherein the eye disorder is wet AMD or an ocular neovascular disease caused by injury to the eye.
